(19) United States
(12) Patent Application Publication
(10) Pub. No.: US 2026/0334890 A1
(43) Pub. Date: Oct. 29, 2026

(54) SYSTEM

(71) Applicant: SoftBank Group Corp., Tokyo (JP)

(72) Inventor: Tatsuya MORIYAMA, Tokyo (JP)

(73) Assignee: SoftBank Group Corp., Tokyo (JP)

(21) Appl. No.: 19/533,430

(22) Filed: Feb. 9, 2026

(30) Foreign Application Priority Data
Feb. 21, 2025 (JP) 2025-027013

(51) Int. Cl. A61B 5/00; A61B 5/11; A61B 5/16; G06F 40/40; G06V 10/77; G06V 10/82; G06V 20/40; G06V 20/52; G06V 40/16; G10L 13/027; G10L 15/02; G10L 25/24; G10L 25/30; G10L 25/63; G10L 25/66; G16H 40/63; G16H 50/20

(52) U.S. Cl. CPC A61B 5/747; A61B 5/0077; A61B 5/1123; A61B 5/165; A61B 5/4803; A61B 5/7257; A61B 5/7267; A61B 5/7278; A61B 5/7282; G06F 40/40; G06V 10/77; G06V 20/40; G06V 20/52; G06V 40/171; G06V 40/174; G10L 13/027; G10L 15/02; G10L 25/24; G10L 25/30; G10L 25/63; G10L 25/66; G16H 40/63; G16H 50/20; A61B 2576/02; G06V 10/82; G06V 2201/03

(57) ABSTRACT

The system according to the embodiment comprises a microphone, a camera, an analysis unit, and a communication unit. The microphone is configured to detect a patient's voice. The camera is configured to detect a patient's facial expression. The analysis unit analyzes data detected by the microphone and the camera and detects abnormalities. The communication unit notifies a nurse center of the details of the abnormality detected by the analysis unit via text and voice.

### CROSS-REFERENCE TO RELATED APPLICATION

The present application claims priority to and incorporates by reference the entire contents of Japanese Patent Application No. 2025-027013 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that hospitalized patients are unable to make a nurse call when their condition suddenly changes or when they are unable to move their bodies.

### SUMMARY OF THE INVENTION

The system according to the embodiment comprises a microphone, a camera, an analysis unit, and a communication unit. The microphone is configured to detect a patient's voice. The camera is configured to detect a patient's facial expression. The analysis unit analyzes data detected by the microphone and the camera and detects abnormalities. The communication unit notifies a nurse center of the details of the abnormality detected by the analysis unit via text and voice.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

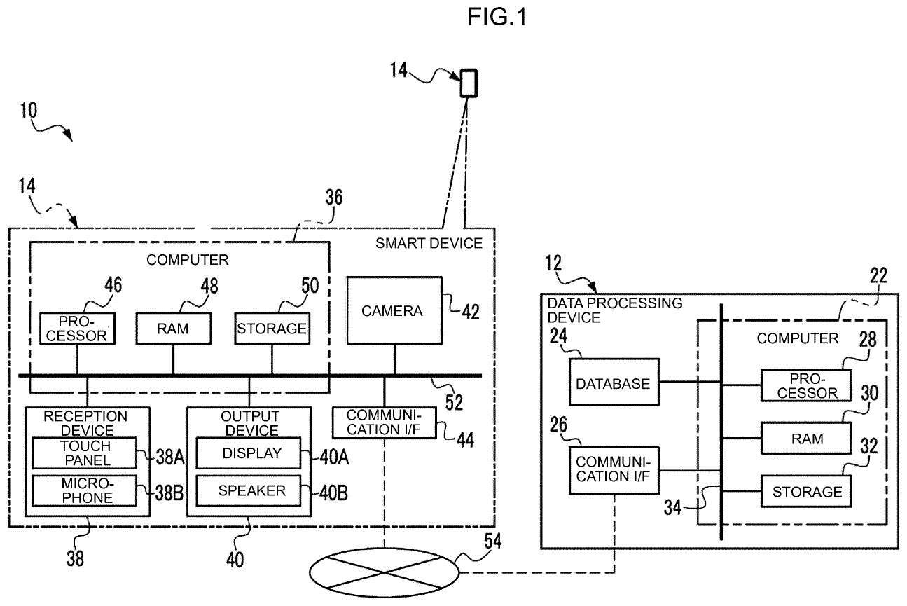

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

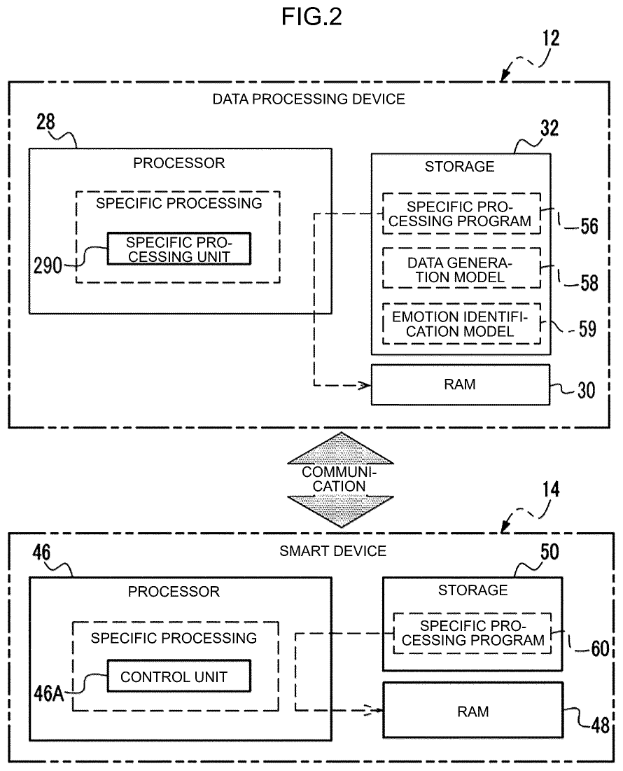

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system designed to solve the problem that hospitalized patients cannot make a nurse call when their condition suddenly deteriorates or when they are unable to move their bodies. This system constantly monitors the patient's condition using sensors that detect subtle voices and facial expressions of the patient. Next, the AI analyzes the detected voice and facial expression data to determine whether an abnormality has occurred. If an abnormality is detected, the AI automatically contacts the nurse center with the details of the abnormality via text and voice. With this system, even in situations where the patient cannot make a nurse call, abnormalities can be detected quickly and appropriate responses can be made. For example, sensors are installed around the bed to detect subtle voices and facial expressions of the patient. This enables real-time monitoring of the patient's sounds and changes in facial expressions. For instance, if the patient groans or shows a painful facial expression, the data is detected by the sensors. The detected data is then sent to the AI for analysis. The AI analyzes changes in the patient's voice and facial expressions to determine whether an abnormality has occurred. For example, the AI can determine whether the patient is experiencing pain or discomfort based on changes in the tone of the patient's voice and facial expressions. If an abnormality is detected, the AI automatically contacts the nurse center with the details of the abnormality via text and voice. For example, content such as “The patient appears to be experiencing pain” is sent to the nurse center via text and voice. This allows nurses to quickly grasp the patient's abnormality and take appropriate action. With this system, even in situations where the patient cannot make a nurse call, abnormalities can be detected quickly and appropriate responses can be made. For example, even if the patient's condition suddenly worsens at night, the sensors detect the abnormality and the AI contacts the nurse center, enabling a prompt response. Furthermore, even if the patient is unable to move, abnormalities can be detected early by monitoring changes in voice and facial expressions. This enables constant monitoring of the patient's condition, rapid detection of abnormalities, and appropriate responses. Specifically, this system uses high-sensitivity microphones and high-resolution cameras installed around the bed to acquire the patient's voice signals (e.g., 1-second voice waveform data sampled at 16 kHz, 512-dimensional MFCC feature vectors) and facial images (e.g., 224×224 pixel RGB image tensors) in real time. The system performs preprocessing such as noise removal, normalization, face region extraction, and voice segment detection on these data, and then inputs them into AI models such as convolutional neural networks (CNN) for voice recognition and Vision Transformers for facial expression recognition. The AI models output labels such as “groaning,” “calling for help,” and “normal conversation” from voice input, and probability distributions such as “pain,” “anxiety,” and “normal” (e.g., pain 0.85, anxiety 0.10, normal 0.05) from facial images. These output values are judged by the abnormality detection module using thresholds (e.g., pain probability of 0.7 or higher), and if judged as abnormal, the abnormality details (e.g., “pain,” “anxiety”) and the supporting scores are generated as structured data. Furthermore, the system converts the abnormality details into natural language text (e.g., “The patient appears to be experiencing pain”) and generates voice data (e.g., WAV format) using a speech synthesis module. These text and voice data are automatically sent to the nurse center terminal, allowing nurses to immediately grasp the patient's abnormal condition. For AI model training, past patient datasets (e.g., thousands of cases with voice, facial expression, and abnormality labels) are used, and loss functions such as cross-entropy loss and Focal Loss are employed to improve accuracy. In addition, multiple AI models (voice model, facial expression model, time-series abnormality detection model) are combined to achieve multifaceted abnormality detection. Subtle changes that are often overlooked by conventional human monitoring can be detected objectively and quickly by high-dimensional feature extraction and rule-based threshold judgment using AI. As a technical effect, this system can detect changes in the patient's condition in real time and with high accuracy compared to conventional nurse call-dependent monitoring, reduce the false detection rate, alleviate the burden on nurses, and greatly improve patient safety. Specific application fields include intensive care units, long-term care wards, and home medical settings, where abnormality detection and early response systems are useful in environments where patients have difficulty expressing their intentions spontaneously.

The patient monitoring system according to the embodiment comprises a microphone, a camera, an analysis unit, and a communication unit. The microphone detects the patient's voice. For example, the microphone can pick up the patient's voice with high sensitivity and detect even subtle sounds. The microphone may be installed, for example, at the head or foot of the patient's bed to monitor the patient's voice in real time. The camera detects the patient's facial expression. For example, the camera can capture the patient's facial expressions in high resolution and detect even subtle changes in facial expressions. The camera may be installed, for example, at the head or foot of the patient's bed to monitor the patient's facial expressions in real time. The analysis unit uses AI to analyze data detected by the microphone and camera and determines abnormalities. For example, the analysis unit analyzes changes in the tone of the patient's voice and facial expressions to determine whether an abnormality has occurred. The analysis unit analyzes changes in the tone of the patient's voice and facial expressions to determine whether the patient is experiencing pain or discomfort. The communication unit uses AI to contact the nurse center with the details of the abnormality determined by the analysis unit via text and voice. For example, when an abnormality is detected, the communication unit sends content such as “The patient appears to be experiencing pain” to the nurse center via text message and voice call. This allows nurses to quickly grasp the patient's abnormality and take appropriate action. Some or all of the above-mentioned processing in the communication unit may be performed using AI or without using AI. For example, the communication unit may use an AI model that takes the abnormality details determined by the analysis unit as input and outputs notifications via text and voice to contact the nurse center. Thus, the patient monitoring system according to the embodiment can constantly monitor the patient's condition, quickly detect abnormalities, and take appropriate action. Specifically, the patient monitoring system installs high-sensitivity MEMS microphones (e.g., 16 kHz, 24 bit, SNR>65 dB) and high-resolution CMOS cameras (e.g., 1920×1080 pixels, 30 fps) at the head and foot of the bed as hardware to acquire voice and image signals in real time. The microphone divides the voice signal into frames (e.g., 1-second units), performs noise reduction and spectral analysis, and extracts acoustic features (e.g., MFCC, Zero Crossing Rate, Spectral Centroid). The camera extracts the face region using a face detection algorithm (e.g., MTCNN) and calculates facial expression features (e.g., Action Units, 68-point facial landmark coordinates). The analysis unit inputs these feature vectors into a convolutional neural network (CNN) for voice abnormality detection, a Vision Transformer for facial expression recognition, or a multimodal fusion model (e.g., Late Fusion, Attention-based Fusion) to perform abnormality judgment. The AI model outputs labels such as “groaning,” “calling for help,” and “normal conversation” from voice input, and probability distributions such as “pain,” “anxiety,” and “normal” from facial expression input. The analysis unit judges these output values as abnormal using threshold judgment (e.g., pain probability of 0.7 or higher) or rule-based logic and sends the abnormality details (e.g., “pain,” “anxiety”) as structured data to the communication unit. The communication unit converts the abnormality details into natural language text (e.g., “The patient appears to be experiencing pain”) and generates voice data using a speech synthesis module (e.g., Tacotron2, WaveNet), which is automatically sent to the nurse center terminal. For AI model training, abnormal voice and facial expression datasets are used, and loss functions such as cross-entropy loss and Focal Loss are employed to improve accuracy. Subtle changes that are often overlooked by conventional human monitoring can be detected objectively and quickly by high-dimensional feature extraction and rule-based threshold judgment using AI. As a technical effect, this system can detect changes in the patient's condition in real time and with high accuracy compared to conventional nurse call-dependent monitoring, reduce the false detection rate, alleviate the burden on nurses, and greatly improve patient safety. Specific application fields include intensive care units, long-term care wards, and home medical settings, where abnormality detection and early response systems are useful in environments where patients have difficulty expressing their intentions spontaneously.

The microphone and camera can be disposed at the head and foot of the bed. For example, the microphone and camera may be installed at the head of the bed to accurately detect the patient's voice and facial expressions. The microphone and camera may also be installed at the foot of the bed. This enables monitoring of the patient's entire body condition. For example, the microphone may be installed at the head of the bed to pick up the patient's voice with high sensitivity. The camera may be installed at the head of the bed to capture the patient's facial expressions in high resolution. Furthermore, the microphone and camera may also be installed at the foot of the bed, enabling monitoring of the patient's entire body condition. This allows for more accurate detection of the patient's voice and facial expressions. Some or all of the above-mentioned processing in the microphone and camera may be performed using AI or without using AI. For example, the microphone and camera can acquire the patient's voice and facial expression data and analyze the data using an AI model. Specifically, the system acquires the patient's speech and breath sounds with high sensitivity using a microphone installed at the head of the bed, and detects lower limb movements and noises on the bed using a microphone installed at the foot of the bed. For the camera, the head camera captures facial expressions and upper body movements in high resolution, while the foot camera captures lower body movements and changes in posture with a wide angle. This enables simultaneous acquisition of voice data (e.g., 16 kHz, 1-second waveform data) and image data (e.g., 1920×1080 pixels, 30 fps video frames) from multiple viewpoints. The AI model synchronizes these multi-viewpoint data in time series and extracts voice features (e.g., MFCC, Spectral Contrast) and image features (e.g., facial landmarks, skeleton estimation vectors). Furthermore, a multimodal fusion model (e.g., Attention-based Fusion) is used to comprehensively analyze abnormal movements and changes in facial expressions of the entire body. For example, if a “painful facial expression” is detected by the head camera and “abnormal leg movement” is detected by the foot camera simultaneously, the AI integrates these and outputs abnormality labels such as “fall risk” or “acute pain” with high accuracy. The output of the AI model is sent to the communication unit as structured data such as abnormality scores (e.g., 0.92) and abnormality types (e.g., “fall risk”). Abnormalities of the entire body that are often overlooked by conventional single-viewpoint monitoring can be detected with greatly improved accuracy and response speed through multi-viewpoint and multimodal AI analysis. As a technical effect, this configuration enables comprehensive monitoring of the patient's entire body condition, improves the accuracy of abnormality detection, and reduces the false detection rate. Application fields include geriatric wards with high fall risk and rehabilitation facilities, where comprehensive body monitoring is required in medical settings.

The analysis unit can analyze changes in the tone of the patient's voice and facial expressions to detect abnormalities. For example, the analysis unit analyzes the tone of the patient's voice to detect abnormalities. The analysis unit can analyze changes in the pitch and volume of the patient's voice to detect abnormal voice patterns. The analysis unit can also analyze changes in the patient's facial expressions to detect abnormalities. For example, the analysis unit can analyze the movement of facial muscles and types of facial expressions to detect abnormal changes in facial expressions. By determining abnormalities from changes in the patient's voice and facial expressions, more rapid responses are possible. Some or all of the above-mentioned processing in the analysis unit may be performed using generative AI or without using generative AI. For example, the analysis unit can input data on changes in the tone of the patient's voice and facial expressions into generative AI to have the generative AI perform abnormality detection. Specifically, the analysis unit receives 1-second voice waveform data sampled at 16 kHz and MFCC feature vectors (e.g., 13 dimensions×100 frames) as voice input, and 224×224 pixel facial images and facial landmark coordinates (e.g., 68 points×2 dimensions) as facial input. For voice analysis, convolutional neural networks (CNN) and recurrent neural networks (RNN) are used to extract features such as pitch (fundamental frequency), volume (RMS energy), and spectral envelope, and detect abnormal voice patterns (e.g., groaning, intermittent screaming, increased silence). For facial expression analysis, Vision Transformers and ResNet-based facial expression recognition models are used to estimate facial muscle movements (e.g., Action Units) and types of facial expressions (e.g., pain, anxiety, normal). The AI model analyzes these features in time series and outputs abnormality scores (e.g., pain 0.85, anxiety 0.10, normal 0.05) and abnormality labels (e.g., “pain”). Abnormality judgment is performed using thresholds (e.g., pain score of 0.7 or higher) or rule-based logic (e.g., simultaneous occurrence of voice abnormality and facial expression abnormality), and the abnormality details are passed as structured data to subsequent processing. For AI model training, abnormal voice and facial expression datasets are used, and loss functions such as cross-entropy loss and Focal Loss are employed to improve accuracy. Subtle changes that are often overlooked by conventional human monitoring can be detected objectively and quickly by high-dimensional feature extraction and rule-based threshold judgment using AI. As a technical effect, the analysis unit achieves improved accuracy of abnormality detection, reduced false detection rate, and enhanced real-time performance, greatly improving patient safety. Application fields include intensive care units, long-term care wards, and home medical settings, where early detection of abnormalities is required in medical settings.

The communication unit can notify the nurse center via text message and voice call when an abnormality is detected. For example, when an abnormality is detected, the communication unit can send content such as “The patient appears to be experiencing pain” to the nurse center via text message. The communication unit can also contact the nurse center via voice call when an abnormality is detected. For example, the communication unit can automatically initiate a voice call when an abnormality is detected and convey the details of the abnormality to the nurse center. This enables prompt contact with the nurse center when an abnormality is detected. Some or all of the above-mentioned processing in the communication unit may be performed using generative AI or without using generative AI. For example, the communication unit can input the details of the abnormality determined by the analysis unit into generative AI and have the generative AI perform notification via text message and voice call. Specifically, the communication unit uses a text generation AI (e.g., large language model) to convert abnormality details (e.g., abnormality labels such as “pain,” “anxiety,” and abnormality scores) received from the analysis unit into natural language text. For example, when input such as “Abnormality type: pain, score: 0.85, basis: groaning voice+pain facial expression” is given, the output is natural Japanese text such as “The patient appears to be experiencing pain. Please check immediately.” Furthermore, for voice call notifications, the text is input into a speech synthesis AI (e.g., Tacotron2, WaveNet) to generate voice data in WAV format. The communication unit automatically sends these text messages to the nurse center terminal and automatically plays the voice data on the nurse call system or smart speaker terminal. The output of the AI model is structured data such as text (e.g., “The patient appears to be experiencing pain”), voice (e.g., synthesized voice file), and notification type (e.g., emergency, normal), which are used for priority judgment and response recording in subsequent nurse response systems. Compared to conventional manual contact and template notifications, automatic generation, diversification, and immediate notification of abnormality details by AI greatly improve the response speed and accuracy of information transmission for nurses. As a technical effect, the communication unit minimizes notification delays when abnormalities occur, reduces the burden on nurses, and enhances patient safety. Application fields include intensive care units, long-term care wards, and home medical settings, where prompt abnormality notification is required in medical settings.

The microphone can estimate the patient's emotion and set the sensitivity of the microphone based on the estimated emotion of the patient. For example, if the patient is feeling anxious, the sensitivity is increased to detect even subtle voices. If the patient is relaxed, the sensitivity can be returned to normal. Furthermore, if the patient is experiencing pain, the sensitivity is set to maximum to reliably detect all voices. By adjusting the sensitivity of the microphone according to the patient's emotion, more accurate voice data can be obtained. Emotion estimation is realized using, for example, an emotion engine or generative AI with emotion estimation functions. Generative AI may be, for example, a text generation AI (such as LLM) or a multimodal generative AI, but is not limited to these examples. Some or all of the above-mentioned processing in the microphone may be performed using AI or without using AI. For example, the microphone can input the patient's voice data into generative AI and have the generative AI perform emotion estimation and sensitivity adjustment. Specifically, the microphone acquires voice waveform data sampled at 16 kHz and MFCC feature vectors in real time and inputs them into an emotion estimation AI (e.g., CNN+RNN model for voice emotion recognition). The AI model extracts features such as pitch, formant, spectral envelope, and speech rate, and outputs emotion labels such as “anxiety,” “relaxation,” and “pain” and their probability distributions (e.g., anxiety 0.75, relaxation 0.15, pain 0.10). Based on the emotion estimation results, the microphone sensitivity control module automatically adjusts gain values and noise filter thresholds. For example, if the anxiety probability is 0.7 or higher, the sensitivity is increased by +10 dB; if the pain probability is 0.8 or higher, the sensitivity is set to maximum. Conversely, in a relaxed state, the sensitivity is returned to normal. For AI model training, emotion-labeled voice datasets (e.g., EmoDB, RAVDESS) are used, and cross-entropy loss is employed as the loss function. The output of the AI model is structured data such as emotion label (e.g., “anxiety”), probability value (e.g., 0.75), and recommended sensitivity setting (e.g., +10 dB), which are used in subsequent voice detection and abnormality judgment modules. Subtle voices that are difficult to detect with conventional fixed-sensitivity microphones can be detected with greatly improved accuracy and abnormality detection speed through AI-based emotion estimation and dynamic sensitivity control. As a technical effect, this configuration enables optimal voice data acquisition according to the patient's emotional state, improving the accuracy of abnormality detection and reducing the false detection rate. Application fields include psychiatric wards and pediatric wards, where emotional changes are signs of abnormality in medical settings.

The microphone can analyze past voice data of the patient and set an appropriate voice pattern for detecting abnormalities. For example, the microphone can learn abnormal voice patterns previously uttered by the patient and judge similar voices as abnormal when detected. The microphone can also record the patient's normal voice tone and rhythm and judge voices that differ from these as abnormal when detected. Furthermore, the microphone can analyze changes in the patient's voice in real time and issue an alert when an abnormal pattern is detected. By analyzing past voice data, abnormalities can be detected more accurately. Some or all of the above-mentioned processing in the microphone may be performed using AI or without using AI. For example, the microphone can input the patient's past voice data into generative AI and have the generative AI set voice patterns for abnormality detection. Specifically, the microphone acquires past voice data accumulated for each patient (e.g., 16 kHz sampled voice waveform, MFCC feature vectors, pitch/rhythm/formant sequences for each utterance) from a database and inputs them as training data into a convolutional neural network (CNN), recurrent neural network (RNN), or self-supervised learning model (e.g., Autoencoder, One-Class SVM) for abnormality detection. The microphone trains the AI model with labeled normal voice patterns (e.g., normal conversation, resting breath sounds) and abnormal voice patterns (e.g., groaning, intermittent screaming, increased silence). The AI model outputs abnormality scores (e.g., 0.92) and abnormality type labels (e.g., “groaning”) from the input voice feature sequences. For example, input examples include “voice waveform data for the past week,” “time-series array of MFCC features,” and “voice clips at abnormal occurrence,” and output examples include “abnormality score 0.85,” “abnormality type: intermittent screaming,” and “difference features from normal voice.” The AI model generates an alert signal when the abnormality score exceeds a threshold (e.g., 0.7) and sends the abnormality details to subsequent communication units or nurse center notification systems. Furthermore, the microphone builds a personalized model for each patient and continuously updates the patient's normal voice pattern to flexibly accommodate individual differences and temporal changes. For AI model training, past abnormal and normal voice datasets are used, and loss functions such as cross-entropy loss and reconstruction error for abnormality detection are employed. Subtle abnormalities that are often overlooked by simple threshold judgment or human auditory monitoring can be detected objectively and quickly by high-dimensional feature extraction and pattern learning using AI. As a technical effect, this configuration achieves improved accuracy of abnormality detection based on patient-specific voice patterns, reduced false detection rate, and enhanced real-time performance, greatly improving patient safety. Specific application fields include long-term care wards, home medical settings, and monitoring of patients with speech disorders, where individual optimization is required in medical settings.

The microphone can be configured so that its installation position enables detection of the patient's breath sounds and heart sounds. For example, the microphone may be installed near the patient's chest to detect breath sounds and heart sounds. The microphone may also be installed at the head and foot of the patient to detect sounds from the entire body. Furthermore, the position of the microphone can be automatically adjusted according to the patient's posture to detect optimal voice signals. By detecting breath sounds and heart sounds, more abnormalities can be detected. Some or all of the above-mentioned processing in the microphone may be performed using AI or without using AI. For example, the microphone can input the patient's breath sounds and heart sounds data into generative AI and have the generative AI perform abnormality detection. Specifically, the microphone installs high-sensitivity MEMS microphones (e.g., 20 Hz-2 kHz bandwidth, SNR>65 dB) at multiple locations such as near the chest and at the head and foot of the bed to acquire breath sounds (e.g., inspiratory/expiratory sound pressure waveforms, spectral features for each breathing cycle) and heart sounds (e.g., S1/S2 peak detection, waveform patterns for each cardiac cycle) in real time. The microphone divides the acquired acoustic signals into frames (e.g., 0.5-second units), performs noise reduction and band-pass filtering, and inputs them into convolutional neural networks (CNN) for breath sound analysis and recurrent neural networks (RNN) for heart sound analysis. The AI model receives input examples such as “1-minute breath sound waveform,” “heart sound peak time series,” and “MFCC feature arrays,” and outputs labels and abnormality scores for abnormal breath sounds (e.g., wheezing, apnea) and abnormal heart sounds (e.g., arrhythmia, heart murmur) (e.g., breath abnormality 0.88, heart sound abnormality 0.92). Furthermore, the patient's posture changes (e.g., turning over, sitting up) are detected by accelerometers or camera images, and the microphone's directivity and gain are automatically adjusted to achieve optimal acoustic signal acquisition. The AI model generates an alert signal when the abnormality score exceeds a threshold (e.g., 0.8) and sends the abnormality details to subsequent analysis units or communication units. For AI model training, abnormal breath sound and heart sound datasets (e.g., PhysioNet/CinC Challenge data) are used, and loss functions such as cross-entropy loss and Focal Loss are applied. Subtle abnormalities that are difficult to detect by conventional auscultation or single-location acoustic monitoring can be detected with greatly improved accuracy and response speed through multi-point and multi-frequency band analysis and pattern recognition using AI. As a technical effect, this configuration achieves improved accuracy of abnormality detection for various biological sounds including breath sounds and heart sounds, reduced false detection rate, and enhanced real-time performance, thereby improving patient safety. Application fields include intensive care units for patients with respiratory or cardiac diseases, home medical care, and rehabilitation facilities, where comprehensive body monitoring is required in medical settings.

The microphone can estimate the patient's emotion and set the recording time of the microphone based on the estimated emotion of the patient. For example, if the patient is feeling anxious, the recording time is extended to avoid missing signs of abnormality. If the patient is relaxed, the recording time can be returned to normal. Furthermore, if the patient is experiencing pain, the recording time is set to maximum to reliably record all voices. By adjusting the recording time according to the patient's emotion, signs of abnormality can be avoided. Emotion estimation is realized using, for example, an emotion engine or generative AI with emotion estimation functions. Generative AI may be, for example, a text generation AI (such as LLM) or a multimodal generative AI, but is not limited to these examples. Some or all of the above-mentioned processing in the microphone may be performed using AI or without using AI. For example, the microphone can input the patient's voice data into generative AI and have the generative AI perform emotion estimation and recording time adjustment. Specifically, the microphone acquires voice waveform data sampled at 16 kHz and MFCC feature vectors in real time and inputs them into convolutional neural networks (CNN) and recurrent neural networks (RNN) for voice emotion recognition, or multimodal AI models (voice+facial expression). The AI model receives input examples such as “1-minute voice waveform,” “MFCC feature array,” and “recent speech clips,” and outputs emotion labels (e.g., anxiety, relaxation, pain) and emotion probability distributions (e.g., anxiety 0.75, relaxation 0.15, pain 0.10). Based on the output of the AI model, the recording control module automatically sets the recording time parameters (e.g., normal 10 seconds, anxiety 30 seconds, pain 60 seconds). For example, if the anxiety probability is 0.7 or higher, the recording time is extended to 30 seconds; if the pain probability is 0.8 or higher, the maximum recording time (e.g., 60 seconds) is set. Conversely, in a relaxed state, the recording time is returned to normal. For AI model training, emotion-labeled voice datasets (e.g., EmoDB, RAVDESS) are used, and cross-entropy loss is applied as the loss function. The output of the AI model is structured data such as emotion label, probability value, and recommended recording time, which are used in subsequent abnormality detection and communication modules. Signs of abnormality that are often overlooked with conventional fixed recording time settings can be detected with greatly improved accuracy and abnormality detection speed through AI-based emotion estimation and dynamic recording control. As a technical effect, this configuration enables optimal voice data acquisition according to the patient's emotional state, improving the accuracy of abnormality detection and reducing the false detection rate. Application fields include psychiatric wards, pediatric wards, and medical settings where emotional changes are signs of abnormality.

The microphone can be provided with a function to automatically set its installation position in accordance with the patient's movement. For example, when the patient turns over, the microphone position is automatically adjusted to detect optimal voice signals. When the patient sits up, the microphone position is automatically adjusted to detect optimal voice signals. Furthermore, when the patient moves, the microphone position is automatically adjusted to detect optimal voice signals. By adjusting the microphone position according to the patient's movement, optimal voice signals can be detected. Some or all of the above-mentioned processing in the microphone may be performed using AI or without using AI. For example, the microphone can input the patient's movement data into generative AI and have the generative AI perform installation position adjustment. Specifically, the microphone acquires acceleration sensor and pressure sensor data placed around the bed, or skeleton estimation vectors (e.g., 17 points, 3D coordinates) extracted from camera images in real time to detect the patient's body movement. The microphone inputs these movement data (e.g., acceleration peaks during turning over, posture change patterns during sitting up) into an AI model (e.g., time-series RNN, Transformer Encoder) to estimate the patient's current posture and type of movement (e.g., supine, lateral, sitting up, sitting on the edge of the bed). The AI model receives input examples such as “10 seconds of acceleration time series,” “skeleton vector array,” and “pressure distribution map,” and outputs “estimated posture: lateral,” “movement type: turning over,” and “recommended microphone position: right side of the head.” Based on the output of the AI model, the microphone automatically adjusts its installation position and directivity (e.g., pan-tilt control, beamforming). For example, when turning over is detected, the microphone is moved to the right side of the head; when sitting up, the microphone switches to collecting sound from above. For AI model training, sensor datasets labeled with body movement are used, and cross-entropy loss and regression error are applied as loss functions. The output of the AI model is structured data such as estimated posture, recommended installation position, and control commands, which are used in subsequent voice detection and abnormality judgment modules. Voice signals during body movement that are difficult to detect with conventional fixed-installation microphones can be detected with greatly improved accuracy and abnormality detection speed through AI-based dynamic installation position control. As a technical effect, this configuration enables optimal voice data acquisition according to the patient's movement, improving the accuracy of abnormality detection and reducing the false detection rate. Application fields include rehabilitation facilities, geriatric wards, and monitoring of patients with frequent body movement.

The microphone can cooperate with other medical devices to improve the accuracy of abnormality detection. For example, the microphone can cooperate with an electrocardiogram to issue an alert when abnormal heart sounds are detected. The microphone can also cooperate with a respiratory monitor to issue an alert when abnormal breath sounds are detected. Furthermore, the microphone can cooperate with a blood pressure monitor to issue an alert when abnormal blood pressure fluctuations are detected. By cooperating with other medical devices, the accuracy of abnormality detection is improved. Some or all of the above-mentioned processing in the microphone may be performed using AI or without using AI. For example, the microphone can input data from other medical devices into generative AI and have the generative AI perform abnormality detection. Specifically, the microphone receives biological signal data (e.g., time-series arrays of heart rate, respiratory rate, and blood pressure values every second) obtained from medical devices such as electrocardiogram (ECG) monitors, respiratory monitors, and blood pressure monitors in real time, and integrates these with simultaneously acquired acoustic data (e.g., heart sound waveforms, breath sound waveforms, acoustic feature vectors) to input into the AI model. The microphone uses a multimodal abnormality detection AI (e.g., Attention-based Fusion model, Graph Neural Network) to learn correlation patterns between multiple biological signals and acoustic signals. The AI model receives input examples such as “10 seconds of ECG waveform+heart sound waveform,” “respiratory rate time series+breath sound spectrum,” and “blood pressure value+abnormal sound occurrence timing,” and outputs “abnormality type (e.g., atrial fibrillation+heart murmur),” “abnormality score (e.g., 0.93),” and “recommended alert content.” The AI model generates an alert signal when the abnormality score exceeds a threshold (e.g., 0.8) and sends the abnormality details to subsequent communication units or nurse center notification systems. For AI model training, abnormality detection datasets integrating multiple medical device data and acoustic data are used, and loss functions such as cross-entropy loss and Focal Loss are applied. Complex abnormalities that are often overlooked by single-sensor abnormality detection can be detected with greatly improved accuracy and response speed through multimodal data integration analysis using AI. As a technical effect, this configuration achieves improved accuracy of abnormality detection through cooperation with multiple medical devices, reduced false detection rate, and enhanced real-time performance, thereby improving patient safety. Application fields include intensive care units, advanced medical settings, and monitoring of patients requiring multiple biological signal monitoring.

The camera can estimate the patient's emotion and set the focal length and exposure of the camera based on the estimated emotion of the patient. For example, if the patient is feeling anxious, the focal length is shortened to capture detailed changes in facial expressions. If the patient is relaxed, the focal length can be returned to normal. Furthermore, if the patient is experiencing pain, the exposure is adjusted to clearly capture changes in facial expressions. By adjusting the camera settings according to the patient's emotion, changes in facial expressions can be captured in detail. Emotion estimation is realized using, for example, an emotion engine or generative AI with emotion estimation functions. Generative AI may be, for example, a text generation AI (such as LLM) or a multimodal generative AI, but is not limited to these examples. Some or all of the above-mentioned processing in the camera may be performed using AI or without using AI. For example, the camera can input the patient's facial expression data into generative AI and have the generative AI perform adjustment of focal length and exposure. Specifically, the camera acquires facial images of the patient (e.g., 1920×1080 pixel RGB images, 30 fps video frames) in real time, performs preprocessing such as face region extraction, brightness normalization, and noise removal, and inputs them into facial expression recognition models such as Vision Transformer or ResNet-based convolutional neural networks. The camera inputs facial images and facial landmark coordinates (e.g., 68 points×2 dimensions) into the AI model, which outputs emotion labels (e.g., “anxiety,” “relaxation,” “pain”) and probability distributions (e.g., anxiety 0.80, relaxation 0.10, pain 0.10). Input examples include “1-second sequence of facial image frames,” “time series of facial landmarks,” and “facial expression feature vectors,” and output examples include “emotion label: anxiety,” “probability value: 0.80,” “recommended focal length: 0.8 m,” and “recommended exposure: +1.5EV.” Based on the output of the AI model, the camera control module automatically adjusts focal length (zoom lens control) and exposure (shutter speed, ISO sensitivity, aperture value). For example, if the anxiety probability is 0.7 or higher, the focal length is set to 0.8 m; if the pain probability is 0.8 or higher, the exposure is increased to +2.0EV to emphasize facial shadows. Conversely, in a relaxed state, the settings are returned to normal (e.g., focal length 1.5 m, exposure 0EV). For AI model training, emotion-labeled facial image datasets (e.g., FER2013, AffectNet) are used, and loss functions such as cross-entropy loss and Focal Loss are applied. The output of the AI model is structured data such as emotion label, probability value, and recommended camera settings (focal length, exposure), which are used in subsequent facial expression analysis and abnormality judgment modules. Subtle changes in facial expressions that cannot be captured by conventional fixed-setting cameras can be detected with greatly improved accuracy and abnormality detection speed through AI-based emotion estimation and dynamic camera control. As a technical effect, this configuration enables optimal image data acquisition according to the patient's emotional state, improving the accuracy of facial expression abnormality detection and reducing the false detection rate. Application fields include psychiatric wards, pediatric wards, medical settings where emotional changes are signs of abnormality, and elderly care facilities where facial expression monitoring is important.

The camera can analyze past facial expression data of the patient and set an appropriate facial expression pattern for detecting abnormalities. For example, the camera can learn abnormal facial expression patterns previously shown by the patient and judge similar facial expressions as abnormal when detected. The camera can also record the patient's normal facial expressions and judge facial expressions that differ from these as abnormal when detected. Furthermore, the camera can analyze changes in the patient's facial expressions in real time and issue an alert when an abnormal pattern is detected. By analyzing past facial expression data, abnormalities can be detected more accurately. Some or all of the above-mentioned processing in the camera may be performed using AI or without using AI. For example, the camera can input the patient's past facial expression data into generative AI and have the generative AI set facial expression patterns for abnormality detection. Specifically, the camera acquires past facial image data accumulated for each patient (e.g., 224×224 pixel facial images, time series of facial landmarks, facial expression feature vectors) from a database and inputs them as training data into Vision Transformer, ResNet-based convolutional neural networks, or self-supervised learning models (e.g., Autoencoder, One-Class SVM) for abnormality detection. The camera trains the AI model with labeled normal facial expression patterns (e.g., normal, relaxed) and abnormal facial expression patterns (e.g., pain, anxiety, fear). The AI model outputs abnormality scores (e.g., 0.91) and abnormality type labels (e.g., “pain”) from the input facial expression feature sequences. Input examples include “facial image frames for the past week,” “time series array of facial landmarks,” and “facial expression clips at abnormal occurrence,” and output examples include “abnormality score 0.85,” “abnormality type: pain facial expression,” and “difference features from normal facial expression.” The AI model generates an alert signal when the abnormality score exceeds a threshold (e.g., 0.7) and sends the abnormality details to subsequent communication units or nurse center notification systems. Furthermore, the camera builds a personalized model for each patient and continuously updates the patient's normal facial expression pattern to flexibly accommodate individual differences and temporal changes. For AI model training, past abnormal and normal facial expression datasets are used, and loss functions such as cross-entropy loss and reconstruction error for abnormality detection are employed. Subtle abnormalities that are often overlooked by simple threshold judgment or human visual monitoring can be detected objectively and quickly by high-dimensional feature extraction and pattern learning using AI. As a technical effect, this configuration achieves improved accuracy of abnormality detection based on patient-specific facial expression patterns, reduced false detection rate, and enhanced real-time performance, greatly improving patient safety. Specific application fields include long-term care wards, home medical settings, and monitoring of patients with facial expression disorders, where individual optimization is required in medical settings.

The camera can be configured so that its installation position enables imaging of the entire body of the patient. For example, the camera may be installed at the head and foot of the patient's bed to enable imaging of the entire body. The camera position can also be automatically adjusted according to the patient's posture to capture optimal images. Furthermore, multiple cameras can be installed around the patient's bed to enable imaging of the entire body. By imaging the entire body of the patient, more abnormalities can be detected. Some or all of the above-mentioned processing in the camera may be performed using AI or without using AI. For example, the camera can input the patient's whole-body video data into generative AI and have the generative AI perform abnormality detection. Specifically, the camera installs high-resolution CMOS cameras (e.g., 1920×1080 pixels, 30 fps) at multiple locations such as the head, foot, and sides of the bed to acquire whole-body video data (e.g., whole-body frame images, video streams) in real time. The camera performs preprocessing such as person detection (e.g., YOLOv5), skeleton estimation (e.g., OpenPose, 17 points, 3D coordinates), and posture classification (e.g., supine, lateral, sitting up), and inputs them into Vision Transformer, 3D CNN, or multimodal fusion models for abnormal movement detection. The AI model receives input examples such as “10 seconds of whole-body video frame sequence,” “time series of skeleton vectors,” and “posture change patterns,” and outputs “abnormal movement label (e.g., fall risk, abnormal posture),” “abnormality score (e.g., 0.92),” and “recommended camera position.” Based on the output of the AI model, the camera automatically adjusts pan, tilt, zoom control, and shooting position to acquire optimal images according to changes in the patient's posture and movement. For example, when turning over is detected, the side camera is activated; when sitting up, the upper camera captures the whole body with a wide angle. For AI model training, whole-body movement-labeled video datasets are used, and cross-entropy loss and regression error are applied as loss functions. The output of the AI model is structured data such as abnormal movement label, abnormality score, and recommended camera position, which are used in subsequent abnormality judgment and communication modules. Whole-body abnormalities that are often overlooked by conventional single-viewpoint cameras can be detected with greatly improved accuracy and response speed through multi-viewpoint and multimodal analysis and dynamic camera control using AI. As a technical effect, this configuration enables comprehensive monitoring of the patient's entire body condition, improves the accuracy of abnormality detection, and reduces the false detection rate. Application fields include geriatric wards with high fall risk and rehabilitation facilities, where comprehensive body monitoring is required in medical settings.

The camera can estimate the patient's emotion and set the imaging frequency of the camera based on the estimated emotion of the patient. For example, if the patient is feeling anxious, the imaging frequency is increased to capture detailed changes in facial expressions. If the patient is relaxed, the imaging frequency can be returned to normal. Furthermore, if the patient is experiencing pain, the imaging frequency is set to maximum to reliably record all facial expressions. By adjusting the imaging frequency according to the patient's emotion, changes in facial expressions can be captured in detail. Emotion estimation is realized using, for example, an emotion engine or generative AI with emotion estimation functions. Generative AI may be, for example, a text generation AI (such as LLM) or a multimodal generative AI, but is not limited to these examples. Some or all of the above-mentioned processing in the camera may be performed using AI or without using AI. For example, the camera can input the patient's facial expression data into generative AI and have the generative AI perform adjustment of imaging frequency. Specifically, the camera acquires facial images (e.g., 1920×1080 pixels, 30 fps) and time-series facial landmark data in real time and inputs them into facial expression recognition models such as Vision Transformer or ResNet-based convolutional neural networks. The AI model receives input examples such as “1-minute sequence of facial image frames,” “facial landmark array,” and “facial expression feature vectors,” and outputs emotion labels (e.g., anxiety, relaxation, pain) and emotion probability distributions (e.g., anxiety 0.75, relaxation 0.15, pain 0.10). Based on the output of the AI model, the imaging control module automatically sets imaging frequency parameters (e.g., normal 1 fps, anxiety 5 fps, pain 10 fps). For example, if the anxiety probability is 0.7 or higher, the imaging frequency is increased to 5 fps; if the pain probability is 0.8 or higher, the maximum imaging frequency (e.g., 10 fps) is set. Conversely, in a relaxed state, the imaging frequency is returned to normal. For AI model training, emotion-labeled facial image datasets (e.g., FER2013, AffectNet) are used, and cross-entropy loss is applied as the loss function. The output of the AI model is structured data such as emotion label, probability value, and recommended imaging frequency, which are used in subsequent facial expression analysis and abnormality judgment modules. Signs of abnormality that are often overlooked with conventional fixed imaging frequency settings can be detected with greatly improved accuracy and abnormality detection speed through AI-based emotion estimation and dynamic imaging control. As a technical effect, this configuration enables optimal image data acquisition according to the patient's emotional state, improving the accuracy of facial expression abnormality detection and reducing the false detection rate. Application fields include psychiatric wards, pediatric wards, and medical settings where emotional changes are signs of abnormality.

The camera can cooperate with other medical devices to improve the accuracy of abnormality detection. For example, the camera can cooperate with an electrocardiogram to issue an alert when abnormal heartbeats are detected. The camera can also cooperate with a respiratory monitor to issue an alert when abnormal breathing patterns are detected. Furthermore, the camera can cooperate with a blood pressure monitor to issue an alert when abnormal blood pressure fluctuations are detected. By cooperating with other medical devices, the accuracy of abnormality detection is improved. Some or all of the above-mentioned processing in the camera may be performed using AI or without using AI. For example, the camera can input data from other medical devices into generative AI and have the generative AI perform abnormality detection. Specifically, the camera receives biological signal data (e.g., time-series arrays of heart rate, respiratory rate, and blood pressure values every second) obtained from medical devices such as electrocardiogram (ECG) monitors, respiratory monitors, and blood pressure monitors in real time, and integrates these with simultaneously acquired video data (e.g., facial images, whole-body video frames, skeleton estimation vectors) to input into the AI model. The camera uses a multimodal abnormality detection AI (e.g., Attention-based Fusion model, Graph Neural Network) to learn correlation patterns between multiple biological signals and video signals. The AI model receives input examples such as “10 seconds of ECG waveform+facial image frames,” “respiratory rate time series+chest movement video,” and “blood pressure value+abnormal facial expression occurrence timing,” and outputs “abnormality type (e.g., atrial fibrillation+pain facial expression),” “abnormality score (e.g., 0.93),” and “recommended alert content.” The AI model generates an alert signal when the abnormality score exceeds a threshold (e.g., 0.8) and sends the abnormality details to subsequent communication units or nurse center notification systems. For AI model training, abnormality detection datasets integrating multiple medical device data and video data are used, and loss functions such as cross-entropy loss and Focal Loss are applied. Complex abnormalities that are often overlooked by single-sensor abnormality detection can be detected with greatly improved accuracy and response speed through multimodal data integration analysis using AI. As a technical effect, this configuration achieves improved accuracy of abnormality detection through cooperation with multiple medical devices, reduced false detection rate, and enhanced real-time performance, thereby improving patient safety. Application fields include intensive care units, advanced medical settings, and monitoring of patients requiring multiple biological signal monitoring.

The camera can be configured to transmit video to the nurse center in real time so that abnormalities can be immediately confirmed when they occur. For example, the camera can transmit video to the nurse center in real time so that abnormalities can be immediately confirmed when they occur. This allows the nurse center to immediately check the patient's condition and respond quickly when an abnormality occurs. Some or all of the above-mentioned processing in the camera may be performed using AI or without using AI. For example, the camera can input real-time video data into generative AI and have the generative AI perform abnormality detection and video transmission. Specifically, the camera acquires facial images and whole-body video frames of the patient (e.g., 1920×1080 pixels, 30 fps) in real time, performs preprocessing such as face region extraction, skeleton estimation, and movement classification, and inputs them into Vision Transformer, 3D CNN, or multimodal AI models for abnormality detection. The AI model receives input examples such as “1-second sequence of facial image frames,” “whole-body video frames,” and “time series of skeleton vectors,” and outputs “abnormality label (e.g., pain facial expression, fall risk),” “abnormality score (e.g., 0.88),” and “video transmission trigger.” Based on the output of the AI model, when the abnormality score exceeds a threshold (e.g., 0.8), the camera automatically transmits the video stream to the nurse center terminal. The video data is transmitted over the network in compressed formats such as H.264 or H.265, allowing the nurse center to immediately check the patient's condition. For AI model training, abnormal facial expression and abnormal movement-labeled video datasets are used, and loss functions such as cross-entropy loss and Focal Loss are applied. The output of the AI model is structured data such as abnormality label, abnormality score, and transmission trigger, which are used for priority judgment and response recording in subsequent nurse response systems. Compared to conventional manual video confirmation and scheduled rounds, automatic abnormality detection and video transmission by AI greatly improve the response speed and accuracy of information transmission for nurses. As a technical effect, this configuration minimizes video confirmation delays when abnormalities occur, reduces the burden on nurses, and enhances patient safety. Application fields include intensive care units, long-term care wards, and home medical settings, where prompt abnormality notification and video confirmation are required in medical settings.

The analysis unit can estimate the patient's emotion and set the analysis algorithm based on the estimated emotion of the patient. For example, if the patient is feeling anxious, the analysis algorithm is set to be sensitive to detect even subtle abnormalities. If the patient is relaxed, the analysis algorithm can be returned to normal. Furthermore, if the patient is experiencing pain, the analysis algorithm is set to maximum to reliably detect all abnormalities. By adjusting the analysis algorithm according to the patient's emotion, even subtle abnormalities can be detected. Emotion estimation is realized using, for example, an emotion engine or generative AI with emotion estimation functions. Generative AI may be, for example, a text generation AI (such as LLM) or a multimodal generative AI, but is not limited to these examples. Some or all of the above-mentioned processing in the analysis unit may be performed using AI or without using AI. For example, the analysis unit can input the patient's emotion data into generative AI and have the generative AI perform adjustment of the analysis algorithm. Specifically, the analysis unit acquires the patient's voice data (e.g., 16 kHz sampled voice waveform, MFCC feature vectors), facial image data (e.g., 224×224 pixel facial images, facial landmark coordinates), and biological signal data (e.g., time-series arrays of heart rate and respiratory rate) in real time and inputs them into emotion estimation AI models (e.g., CNN+RNN for voice emotion recognition, Vision Transformer for facial expression recognition, multimodal fusion models). The analysis unit outputs emotion labels such as “anxiety,” “relaxation,” and “pain” and probability distributions (e.g., anxiety 0.75, relaxation 0.15, pain 0.10) from the AI model. The analysis unit dynamically changes the parameters of the abnormality detection algorithm (e.g., abnormality score threshold, feature extraction window length, ensemble judgment weight coefficient) according to the emotion estimation results. For example, if the anxiety probability is 0.7 or higher, the abnormality score threshold is lowered to 0.6; if the pain probability is 0.8 or higher, all features are analyzed at maximum sensitivity; in a relaxed state, the settings are returned to normal (e.g., threshold 0.8, window length 10 seconds). Input examples for the AI model include “voice waveform for the last minute+facial image frame sequence,” “MFCC feature+time series of facial landmarks,” and “heart rate, respiratory rate+facial expression feature vectors,” and output examples include “emotion label: anxiety,” “probability value: 0.75,” and “recommended threshold: 0.6.” The analysis unit automatically adjusts the parameters of abnormality detection AI (e.g., CNN, RNN, Autoencoder) and rule-based judgment logic based on these outputs to detect even subtle abnormalities (e.g., small groaning, mild pain facial expression) with high sensitivity. For AI model training, emotion-labeled voice, facial expression, and biological signal datasets are used, and loss functions such as cross-entropy loss and Focal Loss are applied. The output of the AI model is structured data such as emotion label, probability value, and recommended algorithm setting, which are used in subsequent abnormality judgment and communication modules. Subtle abnormalities associated with emotional changes that are often overlooked by conventional fixed-parameter abnormality detection can be detected with greatly improved accuracy and abnormality detection speed through AI-based emotion estimation and dynamic algorithm control. As a technical effect, the analysis unit enables optimal abnormality detection according to the patient's emotional state, achieving reduced false detection rate and improved real-time performance. Specific application fields include psychiatric wards, pediatric wards, medical settings where emotional changes are signs of abnormality, and elderly care facilities, where detailed abnormality monitoring according to emotional state is required.

The analysis unit can refer to past data of the patient and perform learning to improve the accuracy of abnormality detection. For example, the analysis unit can learn past voice and facial expression data of the patient to improve the accuracy of abnormality detection. The analysis unit can also refer to past abnormality data of the patient and issue an alert when similar abnormalities are detected. Furthermore, the analysis unit can analyze past data of the patient and learn abnormality patterns to improve detection accuracy. By referring to past data, the accuracy of abnormality detection is improved. Some or all of the above-mentioned processing in the analysis unit may be performed using AI or without using AI. For example, the analysis unit can input the patient's past data into generative AI and have the generative AI perform learning for abnormality detection. Specifically, the analysis unit acquires past voice data (e.g., 16 kHz sampled voice waveform, MFCC feature vectors), facial image data (e.g., 224×224 pixel facial images, time series of facial landmarks), biological signal data (e.g., time-series arrays of heart rate and respiratory rate), and labeled data at abnormal occurrence (e.g., abnormality type, occurrence timing) accumulated for each patient from a database. The analysis unit inputs these past data as training data into convolutional neural networks (CNN), recurrent neural networks (RNN), self-supervised learning models (e.g., Autoencoder, One-Class SVM), or multimodal fusion models for abnormality detection to learn normal and abnormal patterns for each patient. The AI model receives input examples such as “voice waveform for the past week+facial image frames,” “MFCC feature+time series of facial landmarks,” and “heart rate, respiratory rate+features at abnormal occurrence,” and outputs “abnormality score 0.85,” “abnormality type: intermittent screaming,” and “difference features from normal patterns.” Based on the output of the AI model, the analysis unit generates an alert signal when the abnormality score exceeds a threshold (e.g., 0.7) and sends the abnormality details to subsequent communication units or nurse center notification systems. Furthermore, the analysis unit builds a personalized model for each patient and continuously updates the patient's normal patterns to flexibly accommodate individual differences and temporal changes. For AI model training, past abnormal and normal datasets are used, and loss functions such as cross-entropy loss and reconstruction error for abnormality detection are applied. Subtle abnormalities that are often overlooked by simple threshold judgment or human visual/auditory monitoring can be detected objectively and quickly by high-dimensional feature extraction and pattern learning using AI. As a technical effect, the analysis unit achieves improved accuracy of abnormality detection based on past data for each patient, reduced false detection rate, and enhanced real-time performance, greatly improving patient safety. Specific application fields include long-term care wards, home medical settings, and monitoring of patients with speech or facial expression disorders, where individual optimization is required in medical settings.

The analysis unit may have a function to determine the type and urgency of an abnormality when an abnormality is detected. For example, when an abnormality is detected, the analysis unit can determine the type of abnormality and propose an appropriate response. Additionally, when an abnormality is detected, the analysis unit can determine the urgency and issue an alert if a prompt response is required. Furthermore, the analysis unit can comprehensively judge both the type and urgency of the abnormality when an abnormality is detected and propose the optimal response. By determining the type and urgency of the abnormality, appropriate responses can be provided. Some or all of the above-described processes in the analysis unit may be performed using AI, or may be performed without using AI. For example, the analysis unit may input abnormality data into a generative AI and have the generative AI determine the type and urgency of the abnormality. Specifically, the analysis unit receives as input abnormality labels (e.g., “pain,” “fall risk,” “respiratory abnormality”), abnormality scores (e.g., 0.92), and related biosignal data (e.g., time-series arrays of heart rate, respiratory rate, and blood pressure values) output from an abnormality detection AI model (e.g., CNN, RNN, Vision Transformer, multimodal fusion model). In the abnormality type determination module, the analysis unit classifies the type of abnormality based on the abnormality label and feature pattern (e.g., groaning+pain facial expression+increased heart rate). The urgency determination module comprehensively evaluates the magnitude of the abnormality score, duration of abnormality occurrence, simultaneous occurrence of multiple abnormalities, and degree of deviation in vital signs, and outputs an urgency label (e.g., “emergency,” “caution,” “normal”). Examples of input include “abnormality label: fall risk,” “abnormality score: 0.95,” “heart rate: 120 bpm,” “respiratory rate: 30 breaths/min,” and examples of output include “abnormality type: fall risk,” “urgency: emergency,” “recommended response: immediate on-site confirmation.” The analysis unit transmits these determination results as structured data to the communication unit, where they are used for priority determination and response recording in the nurse center and subsequent response systems. For training the AI model, a dataset labeled with abnormality type and urgency is used, and loss functions such as cross-entropy loss and Focal Loss are applied. Complex abnormalities that are often overlooked by conventional simple abnormality detection or human experience-based urgency judgment can be objectively and rapidly addressed by AI-based multidimensional feature analysis and rule-based urgency determination, enabling optimal response proposals. As a technical effect, the analysis unit achieves improved response accuracy through automatic determination of abnormality type and urgency, reduced false detection rate, and enhanced real-time performance, thereby greatly improving patient safety. Specific application fields include intensive care units, advanced medical settings, and patient monitoring environments where emergency response is required.

The analysis unit can estimate the patient's emotion and set the display method of the analysis result based on the estimated emotion of the patient. For example, if the patient is feeling anxious, the analysis unit can display the analysis result simply to reduce visual stress. If the patient is relaxed, detailed analysis results can be displayed to provide information. Furthermore, if the patient is feeling pain, the analysis result can be displayed quickly to prompt appropriate action. By adjusting the display method of the analysis result according to the patient's emotion, visual stress can be reduced and appropriate information can be provided. Emotion estimation may be realized using, for example, an emotion engine or generative AI with emotion estimation functions. Generative AI may include text generation AI (e.g., LLM) or multimodal generative AI, but is not limited to these examples. Some or all of the above-described processes in the analysis unit may be performed using AI, or may be performed without using AI. For example, the analysis unit may input the patient's emotion data into a generative AI and have the generative AI adjust the display method of the analysis result. Specifically, the analysis unit inputs the patient's voice data (e.g., 16 kHz sampled audio waveform, MFCC feature vector), facial image data (e.g., 224×224 pixel face image, facial landmark coordinates), and biosignal data (e.g., time-series arrays of heart rate and respiratory rate) into an emotion estimation AI model (e.g., CNN+RNN for voice emotion recognition, Vision Transformer for facial expression recognition, multimodal fusion model), and outputs emotion labels (e.g., “anxiety,” “relaxation,” “pain”) and probability distributions (e.g., anxiety 0.80, relaxation 0.10, pain 0.10). According to the emotion estimation result, the analysis unit dynamically changes UI parameters of the analysis result display module (e.g., number of display items, color emphasis, font size, display speed). For example, if the anxiety probability is 0.7 or higher, only the main abnormality items are displayed simply with large font and calm colors; in a relaxed state, detailed analysis graphs and time-series data are also displayed. If the pain probability is 0.8 or higher, urgent abnormality content is immediately displayed as a pop-up to prompt rapid response by nurses. Examples of AI model input include “audio waveform of the last 1 minute+sequence of face image frames,” “MFCC features+time-series of facial landmarks,” “heart rate, respiratory rate+facial feature vector,” and examples of output include “emotion label: anxiety,” “probability value: 0.80,” “recommended display mode: simple.” Based on these outputs, the analysis unit automatically adjusts the UI control module to reduce psychological burden on patients and nurses while providing necessary information in the optimal form. For training the AI model, a dataset of voice, facial expression, and biosignal data labeled with emotion is used, and loss functions such as cross-entropy loss and Focal Loss are applied. Consideration for emotional changes, which is difficult with conventional fixed UIs or uniform information display, is greatly improved in terms of information transmission accuracy and psychological safety by AI-based emotion estimation and dynamic UI control. As a technical effect, the analysis unit realizes optimal information provision according to the emotional state of patients and nurses, achieving improved abnormality response accuracy and reduced stress. Application fields include psychiatric wards, pediatric wards, medical settings where emotional changes are signs of abnormality, and elderly care facilities where psychological consideration is important.

The system according to the embodiment is not limited to the above examples and can be variously modified, for example, as follows. Specifically, the system can flexibly change and expand the sensor configuration for patient monitoring, types of AI models, data flow, abnormality detection algorithms, notification methods, user interfaces, and network configuration. For example, regarding sensor configuration, in addition to voice, facial expression, and biosignals, vibration sensors, temperature sensors, humidity sensors, light sensors, pressure sensors, and acceleration sensors can be added to monitor the patient's movements, environment, and physiological state from multiple perspectives. Regarding AI models, the optimal architecture can be selected and combined according to the application and data characteristics, such as convolutional neural networks (CNN), recurrent neural networks (RNN), Vision Transformer, self-supervised learning models, multimodal fusion models, and graph neural networks. Regarding data flow, the entire process from sensor data preprocessing (e.g., noise removal, normalization, feature extraction), input to AI models, abnormality determination, notification generation, transmission to nurse center terminals, and response recording can be flexibly designed. Regarding abnormality detection algorithms, threshold judgment, rule-based judgment, ensemble learning, time-series anomaly detection, and personalized models can be combined to optimize detection accuracy and false detection rate. Regarding notification methods, various channels such as text messages, voice calls, video streams, and smart device integration can be selected. Regarding user interfaces, display and operation systems optimized for each user can be implemented, such as dashboards for nurses, feedback screens for patients, and statistical analysis screens for administrators. Regarding network configuration, local servers, cloud integration, edge AI processing, and distributed databases can be flexibly designed according to the requirements and security policies of the medical site. Through these various modifications and expansions, the system can realize optimal monitoring, abnormality detection, notification, and response according to the needs of the medical site and patient characteristics, and as a technical effect, achieve improved detection accuracy, reduced false detection rate, enhanced real-time performance, reduced operational burden, and improved patient safety. Application fields include intensive care units, long-term care wards, home medical care, rehabilitation facilities, psychiatric wards, pediatric wards, elderly care facilities, and a wide range of medical and nursing care settings.

The patient monitoring system may further include a vibration sensor. The vibration sensor can be attached to the patient's bed and detect the patient's movements. For example, when the patient is about to fall from the bed or makes abnormal movements, the vibration sensor reacts and can detect the abnormality. Data detected by the vibration sensor is sent to the analysis unit, where it is integrated and analyzed together with data from other sensors. This enables detection of abnormalities based on the patient's movements and prompt response. The vibration sensor can issue an alert, for example, when the patient is about to fall from the bed. Additionally, when the patient makes abnormal movements, the vibration sensor can automatically contact the nurse center. Furthermore, the vibration sensor can learn the patient's movement patterns and issue an alert when abnormal movements are detected. Specifically, the patient monitoring system installs high-sensitivity MEMS vibration sensors (e.g., acceleration resolution 0.01 G, sampling rate 100 Hz) under the bed frame or mattress to acquire the patient's body movements and bed vibration patterns in real time. The vibration sensor applies preprocessing such as noise removal, normalization, and peak detection to the acquired time-series data (e.g., 10 seconds of acceleration waveform, vibration spectral features), and inputs it into a convolutional neural network (CNN) or time-series RNN for abnormal movement detection, or a self-supervised learning model (Autoencoder, One-Class SVM). The AI model receives inputs such as “acceleration waveform during turning over,” “vibration pattern when moving to the edge of the bed,” and “peak waveform just before falling,” and outputs such as “abnormal movement label (e.g., fall risk, abnormal body movement)” and “abnormality score (e.g., 0.91).” The analysis unit integrates and analyzes vibration sensor data and data from other sensors (voice, facial expression, vital signs, etc.) using a multimodal fusion model to improve the accuracy of abnormality determination. For training the AI model, a vibration dataset labeled with abnormal movements is used, and loss functions such as cross-entropy loss and reconstruction error are applied. The AI model outputs structured data such as abnormal movement labels, abnormality scores, and recommended alert content, which are used in subsequent communication units and nurse center notification systems. Subtle abnormal movements that are often overlooked by conventional visual monitoring by humans or detection by a single sensor can be detected with much higher accuracy and speed by AI-based high-sensitivity vibration analysis and multimodal integration. As a technical effect, this configuration enables early detection of fall risks and abnormal body movements, reduces false detection rates, and improves real-time performance, thereby enhancing patient safety. Application fields include elderly wards, rehabilitation facilities, and monitoring of patients with high fall risk.

The patient monitoring system may further include a temperature sensor. The temperature sensor can constantly monitor the patient's body temperature and detect abnormal temperature changes. For example, when the patient's body temperature rises or falls rapidly, the temperature sensor reacts and can detect the abnormality. Data detected by the temperature sensor is sent to the analysis unit, where it is integrated and analyzed together with data from other sensors. This enables detection of abnormalities based on changes in the patient's body temperature and prompt response. The temperature sensor can issue an alert, for example, when the patient's body temperature rises rapidly. Additionally, when the patient's body temperature drops rapidly, the temperature sensor can automatically contact the nurse center. Furthermore, the temperature sensor can learn the patient's body temperature patterns and issue an alert when abnormal temperature changes are detected. Specifically, the patient monitoring system uses high-precision thermistor temperature sensors (e.g., resolution 0.01 °C, sampling rate 1 Hz) that can be installed on the patient's skin surface, underarm, or auricle to acquire body temperature data in real time. The temperature sensor applies preprocessing such as noise removal, outlier removal, and moving average to the acquired time-series data (e.g., 24-hour body temperature trend, 5-minute rapid change pattern), and inputs it into an RNN or self-supervised learning model (Autoencoder, One-Class SVM) for abnormal temperature detection. The AI model receives inputs such as “body temperature time-series during fever,” “rapid drop pattern during hypothermia,” and “normal temperature fluctuation range,” and outputs such as “abnormal temperature label (e.g., high fever, hypothermia)” and “abnormality score (e.g., 0.89).” The analysis unit integrates and analyzes temperature sensor data and data from other sensors (voice, facial expression, vital signs, etc.) using a multimodal fusion model to improve the accuracy of abnormality determination. For training the AI model, a temperature dataset labeled with abnormal temperatures is used, and loss functions such as cross-entropy loss and reconstruction error are applied. The AI model outputs structured data such as abnormal temperature labels, abnormality scores, and recommended alert content, which are used in subsequent communication units and nurse center notification systems. Subtle temperature changes that are often overlooked by conventional scheduled temperature measurements or visual monitoring by humans can be detected with much higher accuracy and speed by AI-based high-precision time-series analysis and multimodal integration. As a technical effect, this configuration enables early detection of abnormal temperatures such as fever and hypothermia, reduces false detection rates, and improves real-time performance, thereby enhancing patient safety. Application fields include infectious disease wards, intensive care units, and home medical care settings where temperature changes are important indicators.

The patient monitoring system may further include a humidity sensor. The humidity sensor can constantly monitor the humidity around the patient and detect abnormal humidity changes. For example, when the humidity around the patient rises or falls rapidly, the humidity sensor reacts and can detect the abnormality. Data detected by the humidity sensor is sent to the analysis unit, where it is integrated and analyzed together with data from other sensors. This enables detection of abnormalities based on changes in the humidity around the patient and prompt response. The humidity sensor can issue an alert, for example, when the humidity around the patient rises rapidly. Additionally, when the humidity around the patient drops rapidly, the humidity sensor can automatically contact the nurse center. Furthermore, the humidity sensor can learn the humidity patterns around the patient and issue an alert when abnormal humidity changes are detected. Specifically, the patient monitoring system uses high-precision digital humidity sensors (e.g., resolution 0.1%, sampling rate 1 Hz) that can be installed in the hospital room or around the bed to acquire environmental humidity data in real time. The humidity sensor applies preprocessing such as noise removal, outlier removal, and moving average to the acquired time-series data (e.g., 24-hour humidity trend, rapid rise/fall pattern), and inputs it into an RNN or self-supervised learning model (Autoencoder, One-Class SVM) for abnormal humidity detection. The AI model receives inputs such as “humidity rise pattern during humidifier operation,” “rapid humidity drop during air conditioning failure,” and “normal humidity fluctuation range,” and outputs such as “abnormal humidity label (e.g., high humidity, low humidity)” and “abnormality score (e.g., 0.87).” The analysis unit integrates and analyzes humidity sensor data and data from other sensors (temperature, voice, facial expression, vital signs, etc.) using a multimodal fusion model to improve the accuracy of abnormality determination. For training the AI model, a humidity dataset labeled with abnormal humidity is used, and loss functions such as cross-entropy loss and reconstruction error are applied. The AI model outputs structured data such as abnormal humidity labels, abnormality scores, and recommended alert content, which are used in subsequent communication units and nurse center notification systems. Subtle humidity changes that are often overlooked by conventional scheduled environmental measurements or visual monitoring by humans can be detected with much higher accuracy and speed by AI-based high-precision time-series analysis and multimodal integration. As a technical effect, this configuration enables early detection of abnormal humidity, reduces false detection rates, and improves real-time performance, thereby enhancing patient safety. Application fields include wards for patients with respiratory diseases, infectious disease wards, and home medical care settings where environmental management is important.

The patient monitoring system may further include a light sensor. The light sensor can constantly monitor the intensity of light around the patient and detect abnormal changes in light. For example, when the light around the patient becomes suddenly stronger or darker, the light sensor reacts and can detect the abnormality. Data detected by the light sensor is sent to the analysis unit, where it is integrated and analyzed together with data from other sensors. This enables detection of abnormalities based on changes in light around the patient and prompt response. The light sensor can issue an alert, for example, when the light around the patient becomes suddenly stronger. Additionally, when the light around the patient becomes suddenly darker, the light sensor can automatically contact the nurse center. Furthermore, the light sensor can learn the light patterns around the patient and issue an alert when abnormal changes in light are detected. Specifically, the patient monitoring system uses high-precision digital light sensors (e.g., resolution 1 lux, sampling rate 1 Hz) that can be installed in the hospital room or around the bed to acquire environmental light intensity data in real time. The light sensor applies preprocessing such as noise removal, outlier removal, and moving average to the acquired time-series data (e.g., 24-hour illuminance trend, rapid increase/decrease pattern), and inputs it into an RNN or self-supervised learning model (Autoencoder, One-Class SVM) for abnormal light change detection. The AI model receives inputs such as “rapid illuminance increase at night,” “rapid illuminance drop during power outage,” and “normal illuminance fluctuation range,” and outputs such as “abnormal light label (e.g., strong light, darkness)” and “abnormality score (e.g., 0.88).” The analysis unit integrates and analyzes light sensor data and data from other sensors (temperature, humidity, voice, facial expression, vital signs, etc.) using a multimodal fusion model to improve the accuracy of abnormality determination. For training the AI model, a light dataset labeled with abnormal light is used, and loss functions such as cross-entropy loss and reconstruction error are applied. The AI model outputs structured data such as abnormal light labels, abnormality scores, and recommended alert content, which are used in subsequent communication units and nurse center notification systems. Subtle changes in light that are often overlooked by conventional scheduled environmental measurements or visual monitoring by humans can be detected with much higher accuracy and speed by AI-based high-precision time-series analysis and multimodal integration. As a technical effect, this configuration enables early detection of abnormal light changes, reduces false detection rates, and improves real-time performance, thereby enhancing patient safety. Application fields include wards for patients with sleep disorders, intensive care units, and home medical care settings where environmental light management is important.

The patient monitoring system may further include a voice recognition unit. The voice recognition unit can analyze the patient's voice and estimate the patient's emotion. For example, if the patient is feeling anxious, the voice recognition unit can estimate that emotion and notify the nurse center. If the patient is relaxed, the unit can estimate that emotion and return to normal monitoring mode. Furthermore, if the patient is feeling pain, the unit can estimate that emotion and respond promptly. This enables appropriate responses based on the patient's emotion. The voice recognition unit can analyze the tone and rhythm of the patient's voice to estimate emotion. Additionally, the unit can analyze changes in the patient's voice in real time and issue an alert when abnormal emotions are detected. Furthermore, the voice recognition unit can learn the patient's voice data to improve the accuracy of emotion estimation. Specifically, the voice recognition unit acquires audio waveform data sampled at 16 kHz, MFCC feature vectors, and pitch, rhythm, and formant sequences for each utterance in real time, and inputs them into a convolutional neural network (CNN) for voice emotion recognition, a recurrent neural network (RNN), or a multimodal AI model (voice+facial expression). The AI model receives inputs such as “1 minute of audio waveform,” “MFCC feature array,” and “recent utterance clip,” and outputs such as “emotion label (e.g., anxiety, relaxation, pain)” and “emotion probability distribution (e.g., anxiety 0.75, relaxation 0.15, pain 0.10).” Based on the AI model's output, the voice recognition unit sends the emotion estimation result to the communication unit or nurse center notification system and automatically adjusts the monitoring mode or response priority as needed. For training the AI model, an emotion-labeled voice dataset (e.g., EmoDB, RAVDESS) is used, and loss functions such as cross-entropy loss and Focal Loss are applied. The AI model outputs structured data such as emotion labels, probability values, and recommended response modes, which are used in subsequent abnormality detection and communication modules. Subtle emotional changes that are often overlooked by conventional human auditory judgment or simple acoustic feature determination can be detected with much higher accuracy and speed by AI-based high-dimensional feature extraction and pattern learning. As a technical effect, this configuration enables optimal response according to the patient's emotional state, improves abnormality detection accuracy, and reduces false detection rates. Application fields include psychiatric wards, pediatric wards, and medical settings where emotional changes are signs of abnormality.

The patient monitoring system may further include a facial expression recognition unit. The facial expression recognition unit can analyze the patient's facial expressions and estimate the patient's emotion. For example, if the patient is feeling anxious, the facial expression recognition unit can estimate that emotion and notify the nurse center. If the patient is relaxed, the unit can estimate that emotion and return to normal monitoring mode. Furthermore, if the patient is feeling pain, the unit can estimate that emotion and respond promptly. This enables appropriate responses based on the patient's emotion. The facial expression recognition unit can analyze the movement of the patient's facial muscles and types of facial expressions to estimate emotion. Additionally, the unit can analyze changes in the patient's facial expressions in real time and issue an alert when abnormal emotions are detected. Furthermore, the facial expression recognition unit can learn the patient's facial expression data to improve the accuracy of emotion estimation. Specifically, the facial expression recognition unit acquires 224×224 pixel face images, time-series facial landmark data, and facial expression feature vectors in real time, and inputs them into a Vision Transformer for facial expression recognition, a ResNet-based convolutional neural network, or a multimodal AI model (facial expression +voice). The AI model receives inputs such as “1 minute of face image frame sequence,” “facial landmark array,” and “facial expression feature vector,” and outputs such as “emotion label (e.g., anxiety, relaxation, pain)” and “emotion probability distribution (e.g., anxiety 0.75, relaxation 0.15, pain 0.10).” Based on the AI model's output, the facial expression recognition unit sends the emotion estimation result to the communication unit or nurse center notification system and automatically adjusts the monitoring mode or response priority as needed. For training the AI model, an emotion-labeled face image dataset (e.g., FER2013, AffectNet) is used, and loss functions such as cross-entropy loss and Focal Loss are applied. The AI model outputs structured data such as emotion labels, probability values, and recommended response modes, which are used in subsequent abnormality detection and communication modules. Subtle emotional changes that are often overlooked by conventional human visual judgment or simple facial feature determination can be detected with much higher accuracy and speed by AI-based high-dimensional feature extraction and pattern learning. As a technical effect, this configuration enables optimal response according to the patient's emotional state, improves abnormality detection accuracy, and reduces false detection rates. Application fields include psychiatric wards, pediatric wards, and medical settings where emotional changes are signs of abnormality.

The patient monitoring system may further include a voice feedback unit. The voice feedback unit can estimate the patient's emotion and provide appropriate voice feedback based on the estimated emotion. For example, if the patient is feeling anxious, the voice feedback unit can provide a voice that gives a sense of reassurance. If the patient is relaxed, the unit can provide a voice that maintains relaxation. Furthermore, if the patient is feeling pain, the unit can provide a voice that alleviates pain. By providing voice feedback according to the patient's emotion, the patient's condition can be improved. The voice feedback unit can analyze the tone and rhythm of the patient's voice to estimate emotion. Additionally, the unit can analyze changes in the patient's voice in real time and provide appropriate voice feedback. Furthermore, the voice feedback unit can learn the patient's voice data to improve the accuracy of voice feedback. Specifically, the voice feedback unit inputs the patient's voice data (e.g., 16 kHz sampled audio waveform, MFCC feature vector) and facial expression data (e.g., face image, time-series facial landmarks) into an emotion estimation AI model (e.g., CNN+RNN for voice emotion recognition, Vision Transformer for facial expression recognition, multimodal fusion model), and outputs emotion labels (e.g., “anxiety,” “relaxation,” “pain”) and probability distributions (e.g., anxiety 0.80, relaxation 0.10, pain 0.10). Based on the AI model's output, the voice feedback unit uses a text generation AI (e.g., large language model) to generate feedback messages according to the patient's condition (e.g., “Please rest assured. Staff will be with you shortly.”), and a voice synthesis AI (e.g., Tacotron2, WaveNet) to generate natural voice data (e.g., WAV format). Examples of AI model input include “audio waveform of the last 1 minute+sequence of face image frames,” “MFCC features+time-series of facial landmarks,” and examples of output include “emotion label: anxiety,” “recommended feedback: reassuring voice.” The voice feedback unit automatically plays these voice data on the patient's bedside terminal or smart speaker to reduce the patient's psychological burden. For training the AI model, a dataset of voice and facial expression data labeled with emotion and feedback effect evaluation data is used, and loss functions such as cross-entropy loss and Focal Loss are applied. Individual optimization, which is difficult with conventional fixed voice or manual response, can be realized in real time for each patient by AI-based emotion estimation and dynamic voice generation. As a technical effect, this configuration achieves psychological stabilization through voice feedback according to the patient's emotional state, increased reassurance during abnormal events, and reduced burden on medical staff. Application fields include psychiatric wards, pediatric wards, and long-term care wards where psychological care is important.

The patient monitoring system may further include a facial expression feedback unit. The facial expression feedback unit can estimate the patient's emotion and provide appropriate facial expression feedback based on the estimated emotion. For example, if the patient is feeling anxious, the facial expression feedback unit can display a facial expression that gives a sense of reassurance. If the patient is relaxed, the unit can display a facial expression that maintains relaxation. Furthermore, if the patient is feeling pain, the unit can display a facial expression that alleviates pain. By providing facial expression feedback according to the patient's emotion, the patient's condition can be improved. The facial expression feedback unit can analyze the movement of the patient's facial muscles and types of facial expressions to estimate emotion. Additionally, the unit can analyze changes in the patient's facial expressions in real time and provide appropriate facial expression feedback. Furthermore, the facial expression feedback unit can learn the patient's facial expression data to improve the accuracy of facial expression feedback. Specifically, the facial expression feedback unit inputs the patient's face image (e.g., 224×224 pixels), time-series facial landmarks, and facial expression feature vectors into an emotion estimation AI model (e.g., Vision Transformer for facial expression recognition, ResNet-based CNN, multimodal fusion model), and outputs emotion labels (e.g., “anxiety,” “relaxation,” “pain”) and probability distributions (e.g., anxiety 0.80, relaxation 0.10, pain 0.10). Based on the AI model's output, the facial expression feedback unit automatically generates and displays facial expression animations or illustrations that give a sense of reassurance (e.g., smile, nod, gentle gaze) on a display or robot terminal. Examples of AI model input include “face image frame sequence of the last 1 minute,” “facial landmark array,” and examples of output include “emotion label: anxiety,” “recommended feedback: reassuring facial expression.” The facial expression feedback unit dynamically adjusts display content, animation speed, color emphasis, and other UI parameters according to the patient's condition and emotional changes. For training the AI model, a dataset of face images labeled with emotion and feedback effect evaluation data is used, and loss functions such as cross-entropy loss and Focal Loss are applied. Individual optimization, which is difficult with conventional fixed illustrations or manual response, can be realized in real time for each patient by AI-based emotion estimation and dynamic facial expression generation. As a technical effect, this configuration achieves psychological stabilization through facial expression feedback according to the patient's emotional state, increased reassurance during abnormal events, and reduced burden on medical staff. Application fields include psychiatric wards, pediatric wards, and long-term care wards where psychological care is important.

The patient monitoring system may further include an environment adjustment unit. The environment adjustment unit can estimate the patient's emotion and adjust the environment of the hospital room based on the estimated emotion. For example, if the patient is feeling anxious, the environment adjustment unit can soften the lighting and play music to provide reassurance. If the patient is relaxed, the unit can provide an appropriate environment to maintain that state. Furthermore, if the patient is feeling pain, the environment adjustment unit can adjust the temperature and humidity to alleviate pain. By adjusting the environment according to the patient's emotion, the patient's condition can be improved. The environment adjustment unit can analyze the patient's voice and facial expression data to estimate emotion. Additionally, the unit can adjust lighting, music, temperature, humidity, and other parameters based on the patient's emotion. Furthermore, the environment adjustment unit can learn the patient's data to improve the accuracy of environment adjustment. Specifically, the environment adjustment unit inputs the patient's voice data (e.g., 16 kHz sampled audio waveform, MFCC feature vector), facial image data (e.g., 224×224 pixel face image, time-series facial landmarks), and biosignal data (e.g., time-series arrays of heart rate and respiratory rate) into an emotion estimation AI model (e.g., CNN+RNN for voice emotion recognition, Vision Transformer for facial expression recognition, multimodal fusion model), and outputs emotion labels (e.g., “anxiety,” “relaxation,” “pain”) and probability distributions (e.g., anxiety 0.80, relaxation 0.10, pain 0.10). Based on the AI model's output, the environment adjustment unit automatically adjusts environmental parameters such as lighting control (e.g., color temperature, brightness adjustment), sound control (e.g., healing music playback), air conditioning control (e.g., temperature, humidity settings), and aroma generator control. Examples of AI model input include “audio waveform of the last 1 minute+sequence of face image frames,” “MFCC features+time-series of facial landmarks,” “heart rate, respiratory rate+facial feature vector,” and examples of output include “emotion label: anxiety,” “recommended environment setting: lighting color temperature 3000K, music: healing.” The environment adjustment unit dynamically controls environment settings, adjustment speed, and priority according to the patient's condition and emotional changes. For training the AI model, a dataset of voice, facial expression, and biosignal data labeled with emotion and environment adjustment effect evaluation data is used, and loss functions such as cross-entropy loss and Focal Loss are applied. Individual optimization, which is difficult with conventional manual environment adjustment or fixed settings, can be realized in real time for each patient by AI-based emotion estimation and dynamic environment control. As a technical effect, this configuration achieves psychological stabilization through environment adjustment according to the patient's emotional state, increased reassurance during abnormal events, and reduced burden on medical staff. Application fields include psychiatric wards, pediatric wards, and long-term care wards where psychological care and environment management are important.

The patient monitoring system may further include a vital sign monitoring unit. The vital sign monitoring unit can constantly monitor the patient's vital signs such as heart rate, blood pressure, and respiratory rate, and detect abnormalities. For example, when the patient's heart rate rises or falls rapidly, the vital sign monitoring unit reacts and can detect the abnormality. Data detected by the vital sign monitoring unit is sent to the analysis unit, where it is integrated and analyzed together with data from other sensors. This enables detection of abnormalities based on the patient's vital signs and prompt response. The vital sign monitoring unit can issue an alert, for example, when the patient's heart rate rises rapidly. Additionally, when the patient's blood pressure fluctuates rapidly, the vital sign monitoring unit can automatically contact the nurse center. Furthermore, the vital sign monitoring unit can learn the patient's vital sign patterns and issue an alert when abnormal fluctuations are detected. Specifically, the vital sign monitoring unit acquires biosignal data (e.g., time-series arrays of heart rate, blood pressure, respiratory rate, SpO2 values every second) from medical devices such as ECG monitors, blood pressure monitors, respiratory monitors, and pulse oximeters in real time. The vital sign monitoring unit applies preprocessing such as noise removal, outlier removal, and moving average to the acquired time-series data (e.g., 24-hour heart rate trend, 5-minute rapid fluctuation pattern), and inputs it into an RNN or self-supervised learning model (Autoencoder, One-Class SVM) for abnormal vital sign detection. The AI model receives inputs such as “heart rate time-series during tachycardia,” “blood pressure trend during hypertension,” and “SpO2 fluctuation during hypoxia,” and outputs such as “abnormal vital sign label (e.g., tachycardia, hypertension, hypoxia)” and “abnormality score (e.g., 0.93).” The analysis unit integrates and analyzes vital sign data and data from other sensors (voice, facial expression, temperature, humidity, etc.) using a multimodal fusion model to improve the accuracy of abnormality determination. For training the AI model, a biosignal dataset labeled with abnormal vital signs is used, and loss functions such as cross-entropy loss and reconstruction error are applied. The AI model outputs structured data such as abnormal vital sign labels, abnormality scores, and recommended alert content, which are used in subsequent communication units and nurse center notification systems. Subtle vital sign fluctuations that are often overlooked by conventional scheduled measurements or visual monitoring by humans can be detected with much higher accuracy and speed by AI-based high-precision time-series analysis and multimodal integration. As a technical effect, this configuration enables early detection of abnormal vital signs, reduces false detection rates, and improves real-time performance, thereby enhancing patient safety. Application fields include intensive care units, emergency wards, and home medical care settings where vital sign monitoring is important.

The following is a brief explanation of the processing flow of Example of the Embodiment. Specifically, the system installs various sensors for patient monitoring (e.g., high-sensitivity microphones, cameras for facial expression recognition, vital sign monitors, environmental sensors) around the bed to acquire voice, image, biosignal, and environmental data in real time. Each sensor data undergoes preprocessing (e.g., noise removal, normalization, feature extraction) and is input into AI models (e.g., CNN for voice abnormality detection, Vision Transformer for facial expression recognition, RNN for vital sign analysis, multimodal fusion model). The AI models output labels such as “groaning,” “calling for help,” “normal conversation” from voice input, probability distributions such as “pain,” “anxiety,” “normal” from facial expression input, and abnormality labels such as “tachycardia,” “hypertension,” “hypoxia” from vital sign input. The abnormality determination module analyzes these output values using threshold judgment and rule-based logic, and generates structured data for abnormality content (e.g., “pain,” “fall risk,” “high fever”) and the corresponding score. Furthermore, the communication unit converts the abnormality content into natural language text (e.g., “The patient appears to be in pain”) or voice data (e.g., WAV format) and automatically sends it to the nurse center terminal. For training the AI models, datasets labeled with abnormality for voice, facial expression, and biosignal data are used, and loss functions such as cross-entropy loss and Focal Loss are applied. Subtle abnormalities that are often overlooked by conventional human monitoring or single-sensor dependent systems can be detected with much higher accuracy and speed by AI-based high-dimensional feature extraction and multimodal integrated analysis. As a technical effect, the system achieves real-time and high-precision abnormality detection, reduced false detection rate, reduced nurse burden, and improved patient safety. Application fields include intensive care units, long-term care wards, and home medical care settings where early detection and rapid response to abnormalities are required.

Step 1: The microphone detects the patient's voice. For example, the microphone picks up the patient's voice with high sensitivity and can detect even subtle sounds. The microphone is installed at the head and foot of the patient's bed and monitors the patient's voice in real time. Step 2: The camera detects the patient's facial expression. For example, the camera captures the patient's facial expression in high resolution and can detect even subtle changes in expression. The camera is installed at the head and foot of the patient's bed and monitors the patient's facial expression in real time. Step 3: The analysis unit uses AI to analyze the data detected by the microphone and camera and determines abnormalities. For example, the analysis unit analyzes the tone of the patient's voice and changes in facial expression to determine whether an abnormality has occurred. The analysis unit analyzes changes in the tone of the patient's voice and changes in facial expression to determine whether the patient is experiencing pain or discomfort. Step 4: The communication unit uses AI to notify the nurse center of the details of the abnormality determined by the analysis unit via text and voice. For example, when an abnormality is detected, the communication unit sends the content, such as “The patient appears to be in pain,” to the nurse center via text message and voice call. This enables nurses to quickly grasp the patient's abnormality and take appropriate action. Specifically, in Step 1, the system uses a high-sensitivity MEMS microphone (e.g., 16 kHz, 24 bit, SNR>65 dB) to acquire voice signals in real time, applies noise reduction and spectral analysis, and extracts acoustic features such as MFCC and Zero Crossing Rate. In Step 2, a high-resolution CMOS camera (e.g., 1920×1080 pixels, 30 fps) is used to acquire facial images and changes in expression, and face region extraction and facial landmark detection are performed. In Step 3, these feature vectors are input into a CNN for voice abnormality detection, a Vision Transformer for facial expression recognition, or a multimodal fusion model, and abnormality labels (e.g., “groaning,” “pain facial expression”) and abnormality scores (e.g., 0.85) are output. The analysis unit determines the abnormality content using threshold judgment and rule-based logic, and in Step 4, the communication unit uses a text generation AI (e.g., large language model) and a voice synthesis AI (e.g., Tacotron2, WaveNet) to generate natural language text and voice data, which are automatically sent to the nurse center terminal. For training the AI models, datasets labeled with abnormality for voice and facial expression data are used, and loss functions such as cross-entropy loss and Focal Loss are applied. Subtle abnormalities that are often overlooked by conventional human monitoring or single-sensor dependent systems can be detected with much higher accuracy and speed by AI-based high-dimensional feature extraction and multimodal integrated analysis. As a technical effect, the system achieves real-time and high-precision abnormality detection, reduced false detection rate, reduced nurse burden, and improved patient safety. Application fields include intensive care units, long-term care wards, and home medical care settings where early detection and rapid response to abnormalities are required.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned microphone, camera, analysis unit, and communication unit is implemented by at least one of, for example, a smart device 14 and a data processing apparatus 12. For example, the microphone is implemented by a microphone 38B of the smart device 14 and detects the patient's voice in real time. The camera is implemented by a camera 42 of the smart device 14 and monitors the patient's facial expression in real time. The analysis unit is implemented, for example, by a specific processing unit 290 of the data processing apparatus 12 and analyzes data detected by the microphone and the camera to determine abnormalities. The communication unit is implemented, for example, by the specific processing unit 290 of the data processing apparatus 12 and communicates the details of the abnormality determined by the analysis unit to the nurse center via text and voice. The correspondence between each unit and the device or control unit is not limited to the examples described above and various modifications are possible.

### Second Embodiment

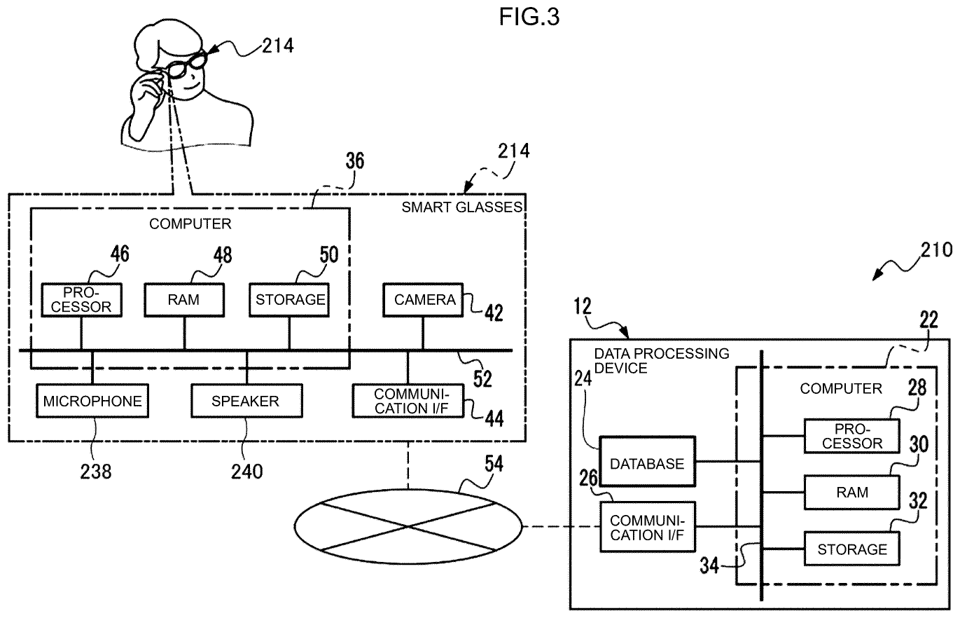

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

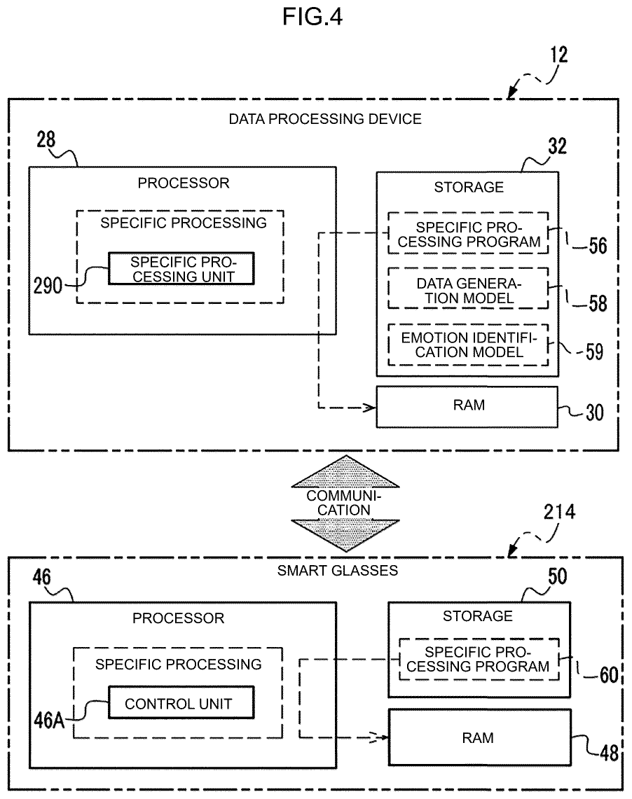

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned microphone, camera, analysis unit, and communication unit is implemented by at least one of, for example, smart glasses 214 and a data processing apparatus 12. For example, the microphone is implemented by a microphone 238 of the smart glasses 214 and detects the patient's voice in real time. The camera is implemented by a camera 42 of the smart glasses 214 and monitors the patient's facial expression in real time. The analysis unit is implemented, for example, by a specific processing unit 290 of the data processing apparatus 12 and analyzes data detected by the microphone and the camera to determine abnormalities. The communication unit is implemented, for example, by the specific processing unit 290 of the data processing apparatus 12 and communicates the details of the abnormality determined by the analysis unit to the nurse center via text and voice. The correspondence between each unit and the device or control unit is not limited to the examples described above and various modifications are possible.

### Third Embodiment

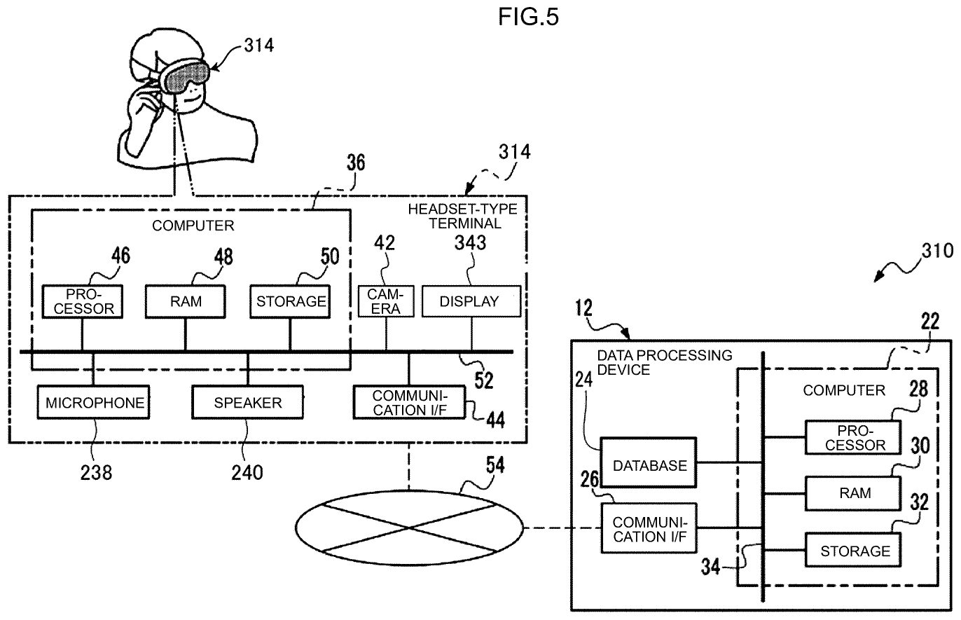

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

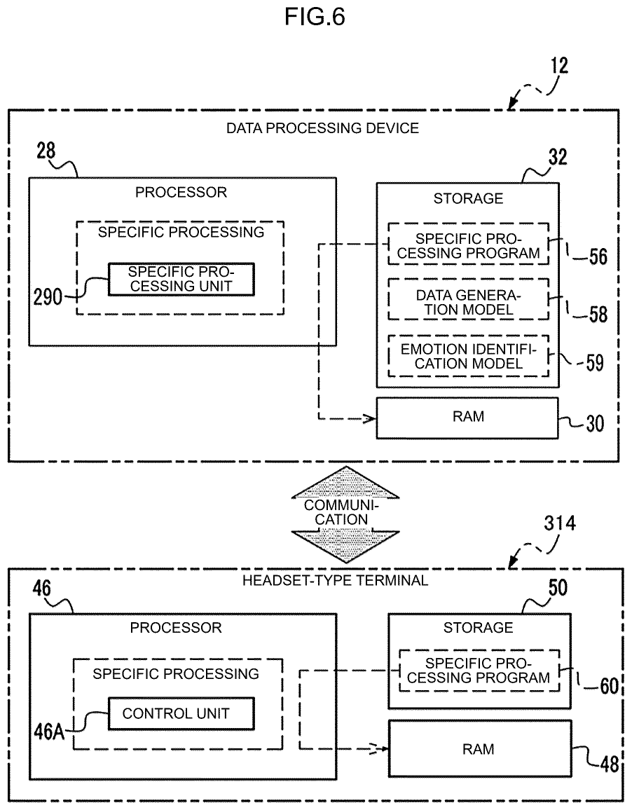

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned microphone, camera, analysis unit, and communication unit is implemented by at least one of, for example, a headset-type terminal 314 and a data processing apparatus 12. For example, the microphone is implemented by a microphone 238 of the headset-type terminal 314 and detects the patient's voice in real time. The camera is implemented by a camera 42 of the headset-type terminal 314 and monitors the patient's facial expression in real time. The analysis unit is implemented, for example, by a specific processing unit 290 of the data processing apparatus 12 and analyzes data detected by the microphone and the camera to determine abnormalities. The communication unit is implemented, for example, by the specific processing unit 290 of the data processing apparatus 12 and communicates the details of the abnormality determined by the analysis unit to the nurse center via text and voice. The correspondence between each unit and the device or control unit is not limited to the examples described above and various modifications are possible.

### Fourth Embodiment

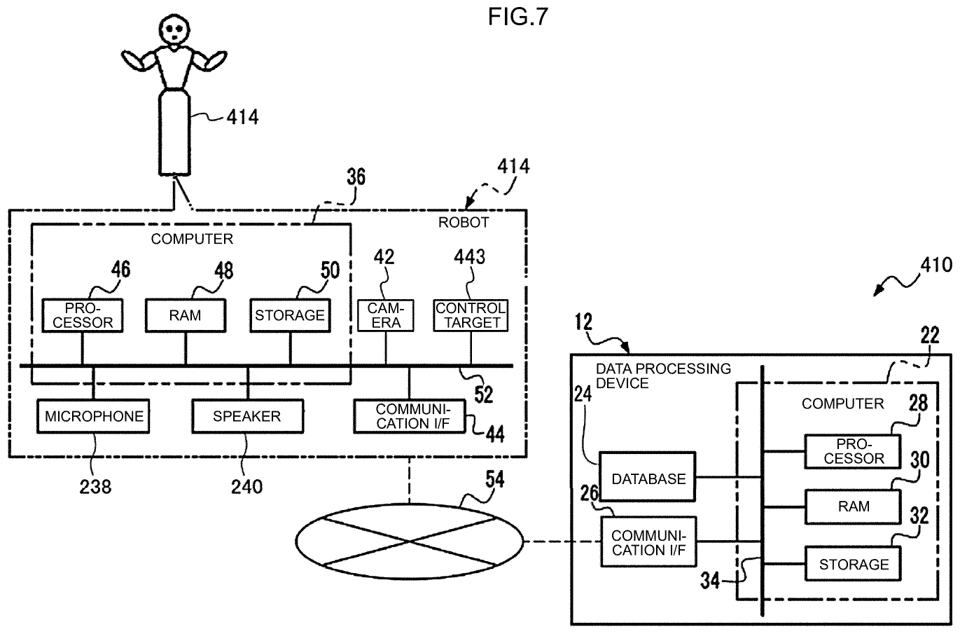

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

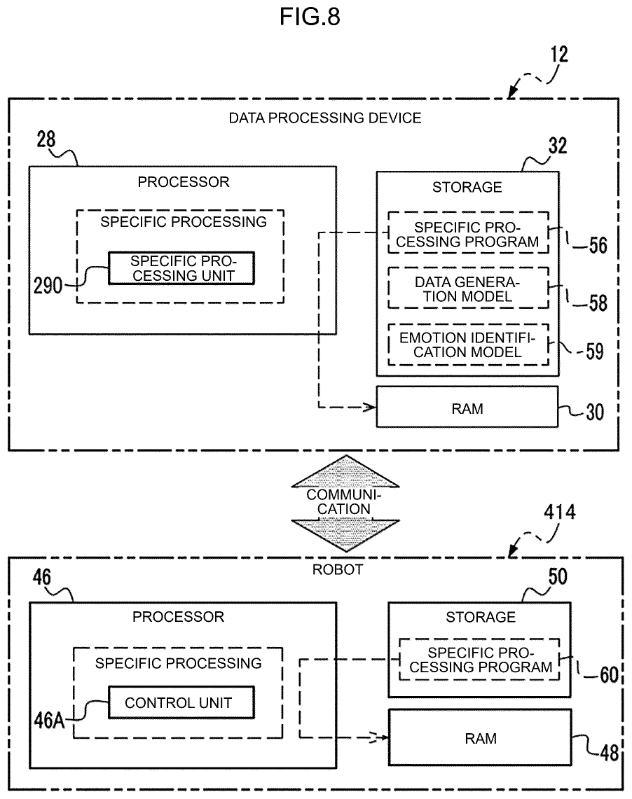

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned microphone, camera, analysis unit, and communication unit is implemented by at least one of, for example, a robot 414 and a data processing apparatus 12. For example, the microphone is implemented by a microphone 238 of the robot 414 and detects the patient's voice in real time. The camera is implemented by a camera 42 of the robot 414 and monitors the patient's facial expression in real time. The analysis unit is implemented, for example, by a specific processing unit 290 of the data processing apparatus 12 and analyzes data detected by the microphone and the camera to determine abnormalities. The communication unit is implemented, for example, by the specific processing unit 290 of the data processing apparatus 12 and communicates the details of the abnormality determined by the analysis unit to the nurse center via text and voice. The correspondence between each unit and the device or control unit is not limited to the examples described above and various modifications are possible.

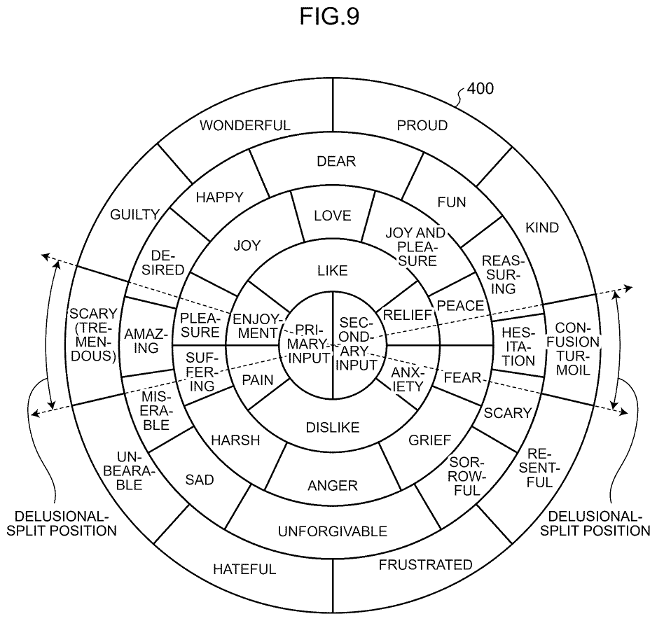

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

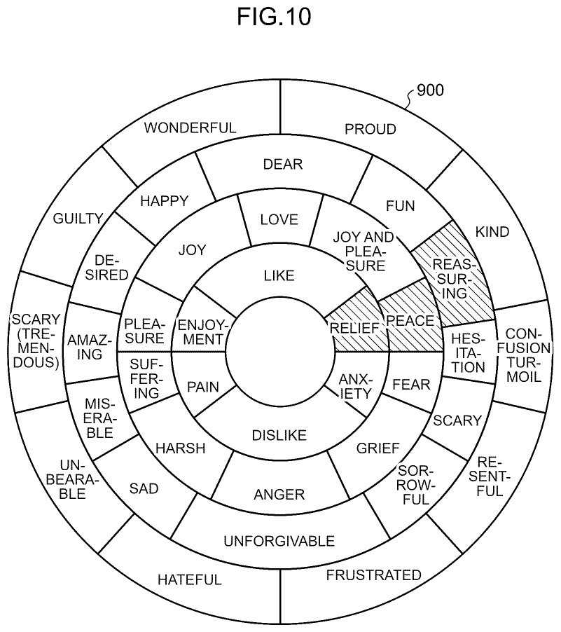

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: a microphone configured to detect a patient's voice; a camera configured to detect a patient's facial expression; an analysis unit configured to analyze data detected by the microphone and the camera and detect abnormalities; and a communication unit configured to notify a nurse center of the details of the abnormality detected by the analysis unit via text and voice.
(Supplementary Note 2) The system according to Supplementary Note 1, wherein the microphone and the camera are disposed at the head and foot of the bed.
(Supplementary Note 3) The system according to Supplementary Note 1, wherein the analysis unit analyzes changes in the tone of the patient's voice and facial expressions to detect abnormalities.
(Supplementary Note 4) The system according to Supplementary Note1, wherein the communication unit notifies the nurse center of an abnormality via text message and voice call when an abnormality is detected.
(Supplementary Note 5) The system according to Supplementary Note 1, wherein the microphone estimates the patient's emotion and sets the sensitivity of the microphone based on the estimated emotion of the patient.
(Supplementary Note 6) The system according to Supplementary Note 1, wherein the microphone analyzes past voice data of the patient and sets an appropriate voice pattern for detecting abnormalities.
(Supplementary Note 7) The system according to Supplementary Note 1, wherein the microphone is configured so that its installation position enables detection of the patient's breath sounds and heart sounds.
(Supplementary Note 8) The system according to Supplementary Note 1, wherein the microphone estimates the patient's emotion and sets the recording time of the microphone based on the estimated emotion of the patient.
(Supplementary Note 9) The system according to Supplementary Note 1, wherein the microphone is provided with a function to automatically set its installation position in accordance with the patient's movement.
(Supplementary Note 10) The system according to Supplementary Note 1, wherein the microphone cooperates with other medical devices to improve the accuracy of abnormality detection.
(Supplementary Note 11) The system according to Supplementary Note 1, wherein the camera estimates the patient's emotion and sets the focal length and exposure of the camera based on the estimated emotion of the patient.
(Supplementary Note 12) The system according to Supplementary Note 1, wherein the camera analyzes past facial expression data of the patient and sets an appropriate facial expression pattern for detecting abnormalities.
(Supplementary Note 13) The system according to Supplementary Note 1, wherein the camera is configured so that its installation position enables imaging of the entire body of the patient.
(Supplementary Note 14) The system according to Supplementary Note 1, wherein the camera estimates the patient's emotion and sets the imaging frequency of the camera based on the estimated emotion of the patient.
(Supplementary Note 15) The system according to Supplementary Note 1, wherein the camera cooperates with other medical devices to improve the accuracy of abnormality detection.
(Supplementary Note 16) The system according to Supplementary Note 1, wherein the camera is configured to transmit video to the nurse center in real time so that abnormalities can be immediately confirmed when they occur.
(Supplementary Note 17) The system according to Supplementary Note 1, wherein the analysis unit estimates the patient's emotion and sets the analysis algorithm based on the estimated emotion of the patient.
(Supplementary Note 18) The system according to Supplementary Note 1, wherein the analysis unit refers to past data of the patient and performs learning to improve the accuracy of abnormality detection.
(Supplementary Note 19) The system according to Supplementary Note 1, wherein the analysis unit has a function to determine the type and urgency of the abnormality when an abnormality is detected.
(Supplementary Note 20) The system according to Supplementary Note 1, wherein the analysis unit estimates the patient's emotion and sets a display method for the analysis result based on the estimated emotion of the patient.

What is claimed is:

1. A system comprising:
a communication interface configured to communicate with a client terminal via a packet-switched network;
a memory storing a data generation model obtained by deep learning on a neural network, and an emotion identification model; and
circuitry configured to:
receive, from the client terminal via the communication interface, audio data captured by a microphone of the client terminal and image data captured by a camera of the client terminal;
extract, using a voice abnormality detection model comprising at least one of a convolutional neural network or a recurrent neural network, acoustic feature vectors from the audio data, the acoustic feature vectors comprising at least one of mel-frequency cepstral coefficients or spectral features;
extract, using a facial expression recognition model comprising at least one of a Vision Transformer or a convolutional neural network, facial expression feature vectors from the image data;
analyze the acoustic feature vectors and the facial expression feature vectors using a multimodal fusion model to generate an abnormality score and an abnormality label;
generate, using the data generation model, notification data comprising at least one of text data or voice data based on the abnormality score and the abnormality label; and
transmit the notification data to a notification terminal via the communication interface and the packet-switched network, the notification data causing the notification terminal to present the notification data to a user.

2. The system according to claim 1, wherein the audio data comprises voice data of a patient, and wherein the abnormality label comprises at least one of a pain indicator, an anxiety indicator, or a distress indicator.

3. The system according to claim 1, wherein the circuitry is further configured to extract the acoustic feature vectors by performing at least one of a short-time Fourier transform, a mel-frequency cepstral coefficient extraction, or a spectral centroid calculation on the audio data.

4. The system according to claim 1, wherein the facial expression recognition model is configured to detect facial landmarks comprising at least 68 coordinate points, and wherein the facial expression feature vectors are generated based on movements of facial muscles derived from the facial landmarks.

5. The system according to claim 1, wherein the multimodal fusion model comprises at least one of an attention-based fusion model or a late fusion model configured to integrate the acoustic feature vectors and the facial expression feature vectors.

6. The system according to claim 1, wherein the circuitry is further configured to estimate an emotion of a subject by applying the emotion identification model to at least one of the audio data or the image data, and to adjust a threshold for the abnormality score based on the estimated emotion.

7. The system according to claim 6, wherein the circuitry is configured to apply a stricter threshold when the estimated emotion indicates anxiety, and to apply a relaxed threshold when the estimated emotion indicates relaxation.

8. The system according to claim 1, wherein the circuitry is further configured to adjust a sensitivity of the voice abnormality detection model based on an estimated emotion, such that when the estimated emotion indicates anxiety, the sensitivity is increased to detect subtle voice changes.

9. The system according to claim 1, wherein the circuitry is further configured to analyze past audio data and past image data associated with a subject to learn normal patterns for the subject, and to detect abnormalities by comparing the acoustic feature vectors and the facial expression feature vectors with the learned normal patterns.

10. The system according to claim 1, wherein the circuitry is further configured to determine a type of the abnormality and an urgency level based on the abnormality score, and to transmit the urgency level together with the notification data to the notification terminal.

11. The system according to claim 10, wherein the urgency level is determined based on at least one of a magnitude of the abnormality score, a duration of abnormality occurrence, or simultaneous occurrence of multiple abnormalities.

12. The system according to claim 1, wherein the circuitry is further configured to receive, from the client terminal via the communication interface, vital sign data comprising at least one of heart rate data, respiratory rate data, or blood pressure data, and to analyze the vital sign data together with the acoustic feature vectors and the facial expression feature vectors to generate the abnormality score.

13. The system according to claim 1, wherein the circuitry is further configured to receive, from the client terminal via the communication interface, vibration data captured by a vibration sensor, and to detect abnormal movements based on the vibration data.

14. The system according to claim 1, wherein the circuitry is further configured to generate the notification data by converting the abnormality label into natural language text using a text generation model, and to generate voice data from the natural language text using a speech synthesis model.

15. The system according to claim 1, wherein the circuitry is further configured to transmit, to the notification terminal via the communication interface, video data captured by the camera of the client terminal when the abnormality score exceeds a predetermined threshold.

16. The system according to claim 1, wherein the circuitry is further configured to estimate an emotion of a subject by applying the emotion identification model to the audio data, and to adjust a display method of the notification data based on the estimated emotion, the display method comprising at least one of a simplified display when the estimated emotion indicates anxiety or a detailed display when the estimated emotion indicates relaxation.

17. The system according to claim 1, wherein the acoustic feature vectors have 128 or more dimensions, and wherein the circuitry is configured to extract the acoustic feature vectors by processing audio frames of the audio data in 1-second units.

18. A system comprising:
a communication interface configured to communicate, via a packet-switched network conforming to at least one of a 5G, Wi-Fi, or Bluetooth communication standard, with a client terminal comprising a microphone and a camera having a CMOS image sensor;
a processor;
a random-access memory;
a memory storing a data generation model obtained by deep learning on a neural network, and an emotion identification model; and
circuitry configured to:
receive, from the client terminal via the communication interface, audio data captured by the microphone and image data captured by the camera having the CMOS image sensor;
extract, using a voice abnormality detection model comprising at least one of a convolutional neural network or a recurrent neural network, acoustic feature vectors from the audio data, the acoustic feature vectors comprising mel-frequency cepstral coefficients;
extract, using a facial expression recognition model comprising a Vision Transformer, facial expression feature vectors from the image data, the facial expression feature vectors comprising facial landmark coordinates;
analyze the acoustic feature vectors and the facial expression feature vectors using a multimodal fusion model to generate an abnormality score and an abnormality label;
estimate an emotion of a subject by applying the emotion identification model to at least one of the audio data or the image data;
generate, using the data generation model, notification data comprising at least one of text data or voice data based on the abnormality score, the abnormality label, and the estimated emotion; and
transmit the notification data to a notification terminal via the communication interface, the notification data causing the notification terminal to present the notification data to a user.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system comprising a communication interface and a memory storing a data generation model obtained by deep learning on a neural network and an emotion identification model, the method comprising:
receiving, from a client terminal via the communication interface and a packet-switched network, audio data captured by a microphone of the client terminal and image data captured by a camera of the client terminal;
extracting, using a voice abnormality detection model comprising at least one of a convolutional neural network or a recurrent neural network, acoustic feature vectors from the audio data, the acoustic feature vectors comprising at least one of mel-frequency cepstral coefficients or spectral features;
extracting, using a facial expression recognition model comprising at least one of a Vision Transformer or a convolutional neural network, facial expression feature vectors from the image data;
analyzing the acoustic feature vectors and the facial expression feature vectors using a multimodal fusion model to generate an abnormality score and an abnormality label;
generating, using the data generation model, notification data comprising at least one of text data or voice data based on the abnormality score and the abnormality label; and
transmitting the notification data to a notification terminal via the communication interface and the packet-switched network, the notification data causing the notification terminal to present the notification data to a user.
